(19) United States
(12) Patent Application Publication
(10) Pub. No.: US 2026/0102401 A1
(43) Pub. Date: Apr. 16, 2026

(54) Combination Therapies

(71) Applicant: MIRATI THERAPEUTICS, INC., PRINCETON, NJ (US)

(72) Inventors: JILL HALLIN, SAN DIEGO, CA (US); PETER OLSON, SAN DIEGO, CA (US)

(73) Assignee: MIRATI THERAPEUTICS, INC., PRINCETON, NJ (US)

(21) Appl. No.: 19/115,906

(22) PCT Filed: Sep. 28, 2023
(86) PCT No.: PCT/US2023/033935

§ 371 Date: Mar. 27, 2025

Related U.S. Application Data
(60) Provisional application No. 63/410,890, filed on Sep. 28, 2022.
(60) Provisional application No. 63/454,208, filed on Mar. 23, 2023.

(51) Int. Cl. A61K 31/519; A61K 31/4184; A61K 31/454; A61K 31/502; A61K 31/5025; A61K 31/55; A61P 35/00

(52) U.S. Cl. CPC A61K 31/519; A61K 31/4184; A61K 31/454; A61K 31/502; A61K 31/5025; A61K 31/55; A61P 35/00

(57) ABSTRACT

The present invention relates to combination therapies for treating KRas G12C cancers. In particular, the present invention relates to methods of treating cancer in a subject in need thereof, comprising administering to the subject a therapeutically effective amount of a combination of a PARP inhibitor and a KRAS G12C inhibitor of Formula (I), Formula (I-A) or Formula (I-B), pharmaceutical compositions comprising a therapeutically effective amounts of the inhibitors, kits comprising the compositions and methods of use therefor.

### FIELD OF THE INVENTION

The present invention relates to combination therapies useful for treating cancer. In particular, the present invention relates to therapeutically effective combinations of an inhibitor of the enzyme poly ADP ribose polymerase (PARP) and a KRas G12C inhibitor, pharmaceutical compositions comprising the inhibitors, kits comprising the compositions and methods of use therefor.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors regulating a wide variety of processes, including cellular proliferation (e.g., see Alamgeer et al., (2013) Current Opin Pharmcol. 13:394-401).

The role of activated KRas in malignancy was observed over thirty years ago (e.g., see Der et al., (1982) Proc. Natl Acad. Sci. USA 79 (11): 3637-3640). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13 (12): 928-942 doi: 10.1038/nrd428). Single nucleotide substitutions that result in missense mutations at codons 12 and 13 of the KRas primary amino acid sequence comprise approximately 40% of these KRas driver mutations in lung adenocarcinoma, with a G12C transversion being the most common activating mutation (e.g., see Dogan et al., (2012) Clin Cancer Res. 18 (22): 6169-6177, published online 2012 Sep. 26. doi: 10.1158/1078-0432.CCR-11-3265).

The well-known role of KRas in malignancy and the discovery of these frequent mutations in KRas in various tumor types made KRas a highly attractable target of the pharmaceutical industry for cancer therapy. Notwithstanding thirty years of large scale discovery efforts to develop inhibitors of KRas for treating cancer, no KRas inhibitor has demonstrated sufficient safety and/or efficacy to obtain regulatory approval (e.g., see McCormick (2015) Clin Cancer Res. 21 (8): 1797-1801).

Despite many failed efforts to target KRas, recent developments in covalent targeting of KRas G12C have shown early promise (e.g., see Ostrem et al., (2013) Nature 503:548-551 and Fell et al., (2018) ACS Med. Chem. Lett. 9:1230-1234) and compounds recently have entered human clinical trials, e.g., AMG510 and MRTX849. Notwithstanding, compounds that inhibit KRas activity are still highly desirable and under investigation, including those that disrupt effectors such as guanine nucleotide exchange factors (e.g., see Sun et al., (2012) Agnew Chem Int Ed Engl. 51 (25): 6140-6143 doi: 10.1002/anie201201358) as well as that target KRas G12C.

While the KRas G12C inhibitors disclosed herein are potent inhibitors of KRas G12C enzymatic activity and exhibit single agent activity inhibiting the in vitro proliferation of cell lines harboring a KRas G12C mutation, the relative potency and/or observed maximal effect of any given KRas G12C inhibitor can vary between KRAS mutant cell lines. The reason or reasons for the range of potencies and observed maximal effect is not fully understood but certain cell lines appear to possess differing intrinsic resistance. Thus, there is a need to develop alternative approaches to maximize the potency, efficacy, therapeutic index and/or clinical benefit of KRas G12C inhibitors in vitro and in vivo.

PARP plays a pivotal role in detecting and repairing single strand breaks in DNA through base excision repair mechanisms. PARP inhibitors compromise the repair pathway by blocking PARP enzymatic activity and convert single-strand breaks to double-strand breaks during replication. Double-strand breaks are typically repaired by the homologous-recombination pathway, which involves BRCA1 and BRCA2 (e.g., O'Connor, (2015) Mol Cell Review 60 (4): 547-560). BRCA1 and BRCA2 genes are tumor suppressor genes, and mutations in BRCA genes confer a high risk for certain cancers and potential resistance to some forms of cancer treatment. When a BRCA gene is mutated, it is no longer effective at repairing broken DNA which helps to prevent onset of certain cancers. These BRCA mutated cancer cells therefore have an increased reliance on PARP to repair DNA strand breaks to enable continued cell division (Bryant et al. (2005) Nature 434:913-917, and Farmer et al. (2005) Nature 434:917-921. The MAPK pathway is one of the most commonly mutated oncogenic pathways in cancer, Deregulation of this pathway is frequently observed and plays a central role in the carcinogenesis and maintenance of several cancers, including melanoma, pancreatic, lung, colorectal, and breast cancers. (e.g., Neuzillet et al., (2014) Pharmacology & Therapeutics 141:160-171).

Several inhibitors exhibiting activity against PARP have been developed, and a number of these inhibitors are or have been inventigated in human clinical trials. Examples of PARP inhibitors suitable for the provided compositions and methods include, but are not limited to olaparib, 4-[[3-[4-(cyclopropanecarbonyl) piperazine-1-carbonyl]-4-fluorophenyl]methyl]-2H-phthalazin-1-one; rucaparib, 6H-Pyrrolo[4,3,2-ef][2]benzazepin-6-one, 8-fluoro-1,3,4,5-tetrahydro-2-[4-[(methylamino)methyl]phenyl]-; niraparib, (S)-2-(4-(piperidin-3-yl)phenyl)-2H-indazole-7-carboxamide; talazoparib, 3H-Pyrido[4,3,2-de]phthalazin-3-one, 5-fluoro-8-(4-fluorophenyl)-2,7,8,9-tetrahydro-9-(1-methyl1H-1,2,4-triazol-5-yl)-, (8S,9R)-; and veliparib, (R)-2-(2-methylpyrrolidin-2-yl)-1H-benzo[d]imidazole-4-carboxamide.

While the PARP inhibitors disclosed herein are potent inhibitors of PARP enzymatic activity and exhibit single agent activity inhibiting the in vitro proliferation of cancer cell lines of the type discussed herein, the relative potency and/or observed maximal effect of any given PARP inhibitor can vary between cancer cell lines. The reason or reasons for the range of potencies and observed maximal effect is not fully understood but certain cell lines appear to possess differing intrinsic resistance. Thus, there is a need to develop alternative approaches to maximize the potency, efficacy, therapeutic index and/or clinical benefit of PARP inhibitors in vitro and in vivo.

### SUMMARY OF THE INVENTION

The combination therapy of the present invention, in one aspect, synergistically increases the potency of KRas G12C inhibitors by coadministering that KRas G12C inhibitor with a PARP inhibitor, resulting in improved efficacy of KRas G12C inhibitors disclosed herein. The combination therapy of the present invention, in another aspect, provides improved clinical benefit to patients compared to treatment with KRas G12C inhibitors disclosed herein as a single agent.

In one aspect of the invention, provided herein are methods of treating cancer in a subject in need thereof, comprising administering to the subject a therapeutically effective amount of a combination of a PARP inhibitor, or a pharmaceutically acceptable salt thereof and a KRAS G12C inhibitor of formula (I):

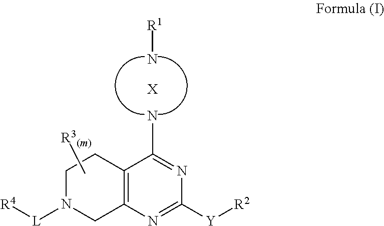

or a pharmaceutically acceptable salt thereof, wherein:
X is a 4-12 membered saturated or partially saturated monocyclic, bridged or spirocyclic ring, wherein the saturated or partially saturated monocyclic ring is optionally substituted with one or more R⁸;
Y is a bond, O, S or NR⁵;
R¹ is —C(O)C(R^{A}) [symbol] C(R^{B})^{p} or —SO₂C(R^{A}) [symbol] C(R^{B})ₚ;
R² is hydrogen, alkyl, hydroxyalkyl, dihydroxyalkyl, alkylaminylalkyl, dialkylaminylalkyl, —Z—NR⁵R¹⁰, heterocyclyl, heterocyclylalkyl, aryl, heteroaryl, or heteroarylalkyl, wherein each of the Z, heterocyclyl, heterocyclylalkyl, aryl, heteroaryl, and heteroarylalkyl may be optionally substituted with one or more R⁹;
Z is C1-C4 alkylene;
each R³ is independently C1-C3 alkyl, oxo, or haloalkyl;
L is a bond, —C(O)—, or C1-C3 alkylene;
R⁴ is hydrogen, cycloalkyl, heterocyclyl, aryl, aralkyl or heteroaryl, wherein each of the cycloalkyl, heterocyclyl, aryl, aralkyl and heteroaryl may be optionally substituted with one or more R⁶ or R⁷;
each R⁵ is independently hydrogen or C1-C3 alkyl;
R⁶ is cycloalkyl, heterocyclyl, heterocyclylalkyl, aryl, or heteroaryl, wherein each of the cycloalkyl, heterocyclyl, aryl, or heteroaryl may be optionally substituted with one or more R⁷;
each R⁷ is independently halogen, hydroxyl, C1-C6 alkyl, cycloalkyl, alkoxy, haloalkyl, amino, cyano, heteroalkyl, hydroxyalkyl or Q-haloalkyl, wherein Q is O or S;
R⁸ is oxo, C1-C3 alkyl, C2-C4 alkynyl, heteroalkyl, cyano, —C(O)OR⁵, —C(O)N(R⁵)₂, —N(R⁵)₂, wherein the C1-C3 alkyl may be optionally substituted with cyano, halogen, —OR⁵, —N(R⁵)₂, or heteroaryl
each R⁹ is independently hydrogen, oxo, acyl, hydroxyl, hydroxyalkyl, cyano, halogen, C1-C6 alkyl, aralkyl, haloalkyl, heteroalkyl, cycloalkyl, heterocyclylalkyl, alkoxy, dialkylaminyl, dialkylamidoalkyl, or dialkylaminylalkyl, wherein the C1-C6 alkyl may be optionally substituted with cycloalkyl;
each R¹⁰ is independently hydrogen, acyl, C1-C3 alkyl, heteroalkyl or hydroxyalkyl;
R¹¹ is haloalkyl;
R^{A} is absent, hydrogen, deuterium, cyano, halogen, C1-C-3 alkyl, haloalkyl, heteroalkyl, —C(O)N(R⁵)₂, or hydroxyalkyl;
each R^{B} is independently hydrogen, deuterium, cyano, C1-C3 alkyl, hydroxyalkyl, heteroalkyl, C1-C3 alkoxy, halogen, haloalkyl, —ZNR⁵R¹¹, —C(O)N(R⁵)₂, —NHC(O)C1-C3 alkyl, —CH₂NHC(O)C1-C3 alkyl, heteroaryl, heteroarylalkyl, dialkylaminylalkyl, or heterocyclylalkyl wherein the heterocyclyl portion is substituted with one or more substituents independently selected from halogen, hydroxyl, alkoxy and C1-C3 alkyl, wherein the heteroaryl or the heteroaryl portion of the heteroarylalkyl is optionally substituted with one or more R⁷;
m is zero or an integer between 1 and 2;
p is one or two; and wherein,
when [symbol] is a triple bond then R^{A} is absent, R^{B} is present and p equals one,
or when [symbol] is a double bond then R^{A} is present, R^{B} is present and p equals two, or R^{A}, R^{B} and the carbon atoms to which they are attached form a 5-8 membered partially saturated cycloalkyl optionally substituted with one or more R⁷.

Also included for use in the methods provided herein are KRas G12C inhibitor compounds of Formula I having the Formula I-A:

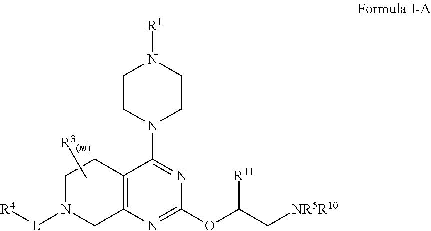

or a pharmaceutically acceptable salt thereof, wherein R¹, R³, R⁴, R⁵, R¹⁰, R¹¹, L and m are as defined for Formula I, and the piperazinyl ring is optionally substituted with R⁸ wherein R⁸ is as defined for Formula I.

Also included for use in the methods provided herein are KRas G12C inhibitor compounds of Formula I having the Formula I-B:

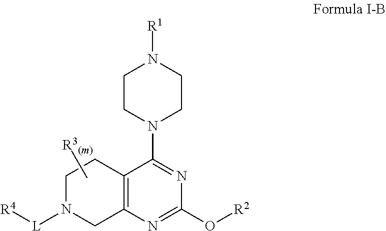

or a pharmaceutically acceptable salt thereof, wherein R¹, R³, R⁴, L and m are as defined for Formula I, R² is heterocyclylalkyl optionally substituted with one or more R⁹ where R⁹ is as defined for Formula I, and the piperazinyl ring is optionally substituted with R⁸, where R⁸ is as defined for Formula I.

Also included for use in the methods provided herein are KRas G12C inhibitor compounds selected from the group consisting of:

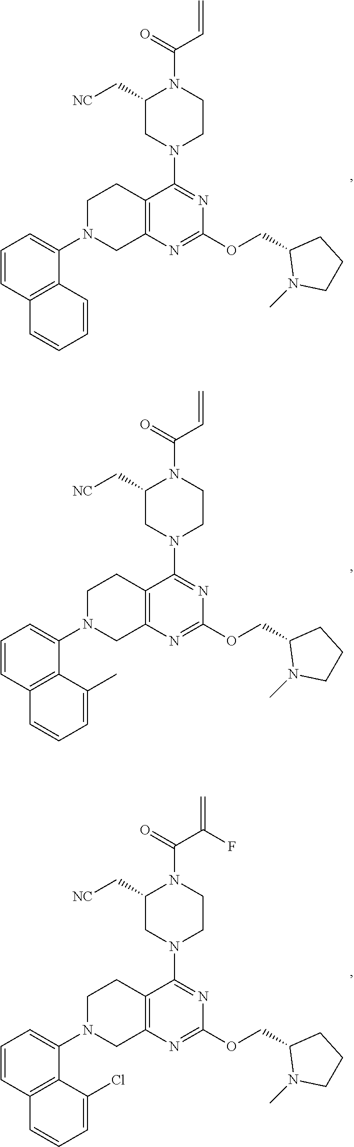 [SMILES: C=CC(=O)N1CCN(c2nc(OC[C@@H]3CCCN3C)nc3c2CCN(c2cccc4ccccc24)C3)C[C@@H]1CC#N ; C=CC(=O)N1CCN(c2nc(OC[C@@H]3CCCN3C)nc3c2CCN(c2cccc4cccc(C)c24)C3)C[C@@H]1CC#N ; C=C(F)C(=O)N1CCN(c2nc(OC[C@@H]3CCCN3C)nc3c2CCN(c2cccc4cccc(Cl)c24)C3)C[C@@H]1CC#N]

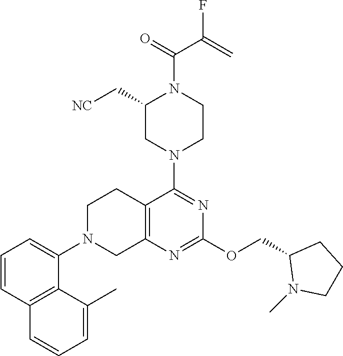 [SMILES: C=C(F)C(=O)N1CCN(c2nc(OC[C@@H]3CCCN3C)nc3c2CCN(c2cccc4cccc(C)c24)C3)C[C@@H]1CC#N]

and pharmaceutically acceptable salts thereof.

In another aspect of the invention, pharmaceutical compositions are provided for use in the methods comprising a therapeutically effective amount of a combination of a PARP inhibitor, or a pharmaceutically acceptable salt thereof, and a KRas G12C inhibitor compound of Formula I, Formula I-A, Formula 1-B, or selected from these aforementioned chemical species (i.e., the species in the preceding paragraph) or a pharmaceutically acceptable salt thereof and a pharmaceutically acceptable excipient.

In one aspect of the invention, provided herein are methods of treating cancer in a subject in need thereof, comprising administering to the subject a therapeutically effective amount of a combination of a PARP inhibitor or a pharmaceutically acceptable salt thereof and a KRAS G12C inhibitor of Formula (I), Formula I-A, Formula I-B, or selected from the aforementioned chemical species or a pharmaceutically acceptable salt thereof. In one embodiment, the cancer is a KRas G12C-associated cancer. In one embodiment, the KRas G12C-associated cancer is lung cancer.

In some aspects of the invention, KRas G12C inhibitor compounds and PARP inhibitors are the only active agents in the provided compositions and methods.

Examples of PARP inhibitors suitable for the provided compositions and methods include, but are not limited to: olaparib, 4-[[3-[4-(cyclopropanecarbonyl) piperazine-1-carbonyl]-4-fluorophenyl]methyl]-2H-phthalazin-1-one; rucaparib, 6H-Pyrrolo[4,3,2-ef][2]benzazepin-6-one, 8-fluoro-1,3,4,5-tetrahydro-2-[4-[(methylamino)methyl]phenyl]-; niraparib, (S)-2-(4-(piperidin-3-yl)phenyl)-2H-indazole-7-carboxamide; talazoparib, 3H-Pyrido[4,3,2-de]phthalazin-3-one, 5-fluoro-8-(4-fluorophenyl)-2,7,8,9-tetrahydro-9-(1-methyl1H-1,2,4-triazol-5-yl)-, (8S,9R)—; and veliparib, (R)-2-(2-methylpyrrolidin-2-yl)-1H-benzo[d]imidazole-4-carboxamide. Other PARP inhibitors suitable for the provided compositions and methods include, but are not limited to: RBN-2397 (Ribon Therapeutics), I-1 (Gu, et al, J med Chem, 2023), KMR-206 (Sanderson, et al, Cell Chemical Biology, 2023), RP14042 (Viswanadha, et al, European Journal of Cancer, 2022) and JAB-26766 (Jacobio).

In yet another aspect, the invention provides for methods for increasing the sensitivity of a cancer cell to a KRas G12C inhibitor, comprising contacting the cancer cell with a therapeutically effective amount of a combination of a KRas G12C inhibitor compound of Formula (I), Formula I-A, Formula I-B, or selected from the aforementioned chemical species and a PARP inhibitor, or pharmaceutically acceptable salts or pharmaceutical compositions thereof, wherein the PARP inhibitor synergistically increases the sensitivity of the cancer cell to the KRas G12C inhibitor. In one embodiment, the contacting is in vitro. In one embodiment, the contacting is in vivo.

Also provided herein are methods for treating cancer in a subject in need thereof, the method comprising (a) determining that cancer is associated with a KRas G12C mutation (e.g., a KRas G12C-associated cancer) (e.g., as determined using a regulatory agency-approved, e.g., FDA-approved, assay or kit); and (b) administering to the patient a therapeutically effective amount of a combination of a PARP inhibitor and a KRas G12C inhibitor compound of Formula I, Formula I-A, Formula 1-B, or selected from the aforementioned chemical species, or pharmaceutically acceptable salts or pharmaceutical compositions thereof, wherein the PARP inhibitor synergistically increases the sensitivity of the KRas G12C-associated cancer to the KRas G12C inhibitor.

Also provided herein are kits comprising a PARP inhibitor and a KRas G12C inhibitor compound of Formula (I), Formula I-A, Formula I-B, or selected from one of the aforementioned species. Also provided is a kit comprising a PARP inhibitor and a KRas G12C inhibitor compound of Formula (I), Formula I-A, Formula I-B, or selected from one of the aforementioned species for use in treating a KRas G12C cancer.

In a related aspect, the invention provides a kit containing a dose of a PARP inhibitor and a KRas G12C inhibitor compound of Formula (I), Formula I-A, Formula I-B or selected from one of the aforementioned species, in an amount effective to inhibit proliferation of cancer cells in a subject. The kit in some cases includes an insert with instructions for administration of a PARP inhibitor and a KRas G12C inhibitor compound of Formula (I), Formula I-A, Formula I-B or selected from one of the aforementioned species. The insert may provide a user with one set of instructions for using the a PARP inhibitor in combination with a KRas G12C inhibitor compound of Formula (I), Formula I-A, Formula I-B, or selected from one of the aforementioned species.

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### BRIEF DESCRIPTION OF THE DRAWINGS

FIG. 1 is a chart demonstrating the effect of administration of compound 478 and/or olaparib on H2122 tumor volumes.

### DETAILED DESCRIPTION OF THE INVENTION

The present invention relates to combination therapies for treating KRas G12C cancers. In particular, the present invention relates to methods of treating cancer in a subject in need thereof, comprising administering to the subject a therapeutically effective amount of a combination of a PARP inhibitor, or a pharmaceutically acceptable salt or a pharmaceutical composition thereof, and a KRAS G12C inhibitor of Formula (I), Formula I-A or Formula I-B, or a pharmaceutically acceptable salt or a pharmaceutical composition thereof, pharmaceutical compositions comprising therapeutically effective amounts of the inhibitors, kits comprising the compositions and methods of use therefor.

Combinations a PARP inhibitors with a KRas G12C inhibitor compounds of Formula (I), Formula I-A or Formula I-B, or a pharmaceutically acceptable salt thereof synergistically increase the potency of KRas G12C inhibitor compounds of Formula (I), Formula I-A or Formula I-B, or a pharmaceutically acceptable salt or a pharmaceutical composition thereof, against cancer cells that express KRas G12C thereby increasing the efficacy and therapeutic index of KRas G12C inhibitor compounds of Formula (I), Formula I-A or Formula I-B, or a pharmaceutically acceptable salt or a pharmaceutical composition thereof.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variant p.Gly 12Cys.

As used herein, a “KRas G12C inhibitor” refers to compounds of the present invention that are represented by Formula (I), Formula I-A and Formula I-B as described herein. These compounds are capable of negatively modulating or inhibiting all or a portion of the enzymatic activity of KRas G12C. The KRas G12C inhibitors of the present invention interact with and irreversibly bind to KRas G12C by forming a covalent adduct with the sulfhydryl side chain of the cysteine residue at position 12 resulting in the inhibition of the enzymatic activity of KRas G12C. In one embodiment, the KRas G12C inhibitor is a compound selected from compound Nos 1-678 (as numbered in WO2019099524), or pharmaceutically acceptable salts thereof (e.g., Example Nos 234, 359, 478 or 507, or a pharmaceutically acceptable salt thereof). In another embodiment, the KRas G12C inhibitor is a compound selected from compound Nos 1-228 (as numbered in US2019-0270743), or pharmaceutically acceptable salts thereof.

A “KRas G12C-associated disease or disorder” as used herein refers to diseases or disorders associated with or mediated by or having a KRas G12C mutation. A non-limiting example of a KRas G12C-associated disease or disorder is a KRas G12C-associated cancer.

As used herein, “PARP” refers to poly (ADP-ribose) polymerase, a family of proteins involved in a number of cellular processes such as DNA repair, genomic stability, and programmed cell death. The PARP family comprises 17 members (10 putative). They vary greatly in structure and function within the cell. PARP1, PARP2, VPARP (PARP4), Tankyrase-1 and -2 (PARP-5a or TNKS, and PARP-5b or TNKS2) have a confirmed PARP activity. Others include PARP3, PARP6, TIPARP (or “PARP7”), PARP8, PARP9, PARP10, PARP11, PARP12, PARP14, PARP15, and PARP16. PARP is composed of four domains of interest: a DNA-binding domain, a caspase-cleaved domain (see below), an auto-modification domain, and a catalytic domain. The DNA-binding domain is composed of two zinc finger motifs. In the presence of damaged DNA (base pair-excised), the DNA-binding domain will bind the DNA and induce a conformational shift. It has been shown that this binding occurs independent of the other domains. This is integral in a programmed cell death model based on caspase cleavage inhibition of PARP. The auto-modification domain is responsible for releasing the protein from the DNA after catalysis. Also, it plays an integral role in cleavage-induced inactivation. The main role of PARP (found in the cell nucleus) is to detect and initiate an immediate cellular response to metabolic, chemical, or radiation-induced single-strand DNA breaks (SSB) by signaling the enzymatic machinery involved in the SSB repair. Once PARP detects a SSB, it binds to the DNA, undergoes a structural change, and begins the synthesis of a polymeric adenosine diphosphate ribose (poly (ADP-ribose) or PAR) chain, which acts as a signal for the other DNA-repairing enzymes. Target enzymes include DNA ligase III (LigIII), DNA polymerase beta (polβ), and scaffolding proteins such as X-ray cross-complementing gene 1 (XRCC1). After repairing, the PAR chains are degraded via Poly(ADP-ribose) glycohydrolase (PARG). NAD+ is required as substrate for generating ADP-ribose monomers. It has been thought that overactivation of PARP may deplete the stores of cellular NAD+ and induce a progressive ATP depletion and necrotic cell death, since glucose oxidation is inhibited. But more recently it was suggested that inhibition of hexokinase activity leads to defects in glycolysis (Andrabi, PNAS 2014). Basal PARP activity also regulates basal bioenergetics. Note below that PARP is inactivated by caspase-3 cleavage during programmed cell death. PARP enzymes are essential in a number of cellular functions, including expression of inflammatory genes: PARP1 is required for the induction of ICAM-1 gene expression by cardiac myocytes and smooth muscle cells, in response to TNF.

As used herein, a “PARP inhibitor” refers to a compound that is capable of negatively modulating or inhibiting all or a portion of the enzymatic activity of a PARP enzyme. More specifically, PARP inhibitors are a group of pharmacological inhibitors of the enzyme poly ADP ribose polymerase (PARP). They are developed for multiple indications, including the treatment of heritable cancers. Several forms of cancer are more dependent on PARP than regular cells, making PARP (PARP1, PARP2 etc) an attractive target for cancer therapy. PARP inhibitors appear to improve progression-free survival in women with recurrent platinum-sensitive ovarian cancer, as evidenced mainly by olaparib added to conventional treatment. In addition to their use in cancer therapy, PARP inhibitors are considered a potential treatment for acute life-threatening diseases, such as stroke and myocardial infarction, as well as for long-term neurodegenerative diseases.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having a KRas G12C mutation (e.g., as determined using a regulatory agency-approved, e.g., FDA-approved, assay or kit). In some embodiments, the subject has a tumor that is positive for a KRas G12C mutation (e.g., as determined using a regulatory agency-approved assay or kit). The subject can be a subject with a tumor(s) that is positive for a KRas G12C mutation (e.g., identified as positive using a regulatory agency-approved, e.g., FDA-approved, assay or kit). The subject can be a subject whose tumors have a KRas G12C mutation (e.g., where the tumor is identified as such using a regulatory agency-approved, e.g., FDA-approved, kit or assay). In some embodiments, the subject is suspected of having a KRas G12C gene-associated cancer. In some embodiments, the subject has a clinical record indicating that the subject has a tumor that has a KRas G12C mutation (and optionally the clinical record indicates that the subject should be treated with any of the compositions provided herein).

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First LR. Pediatric Medicine, 2nd Ed. Baltimore: Williams & Wilkins; 1994.

In some embodiments of any of the methods or uses described herein, an assay is used to determine whether the patient has KRas G12C mutation using a sample (e.g., a biological sample or a biopsy sample such as a paraffin-embedded biopsy sample) from a patient (e.g., a patient suspected of having a KRas G12C-associated cancer, a patient having one or more symptoms of a KRas G12C-associated cancer, and/or a patient that has an increased risk of developing a KRas G12C-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR, quantitative real-time RT-PCR, allele-specific genotyping or ddPCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

The term “amino” refers to —NH₂;

The term “acyl” refers to —C(O)CH₃.

The term “alkyl” as employed herein refers to straight and branched chain aliphatic groups having from 1 to 12 carbon atoms, 1-8 carbon atoms 1-6 carbon atoms, or 1-3 carbon atoms which is optionally substituted with one, two or three substituents. Examples of alkyl groups include, without limitation, methyl, ethyl, propyl, isopropyl, butyl, isobutyl, sec-butyl, tert-butyl, pentyl, and hexyl.

The term “haloalkyl” refers to an alkyl chain in which one or more hydrogen has been replaced by a halogen. Examples of haloalkyls are trifluoromethyl, difluoromethyl and fluoromethyl.

The term “haloalkyloxy” refers to —O-haloalkyl.

An “alkylene,” group is an alkyl group, as defined hereinabove, that is positioned between and serves to connect two other chemical groups. Exemplary alkylene groups include, without limitation, methylene, ethylene, propylene, and butylene.

The term “alkoxy” refers to —OC1-C6 alkyl.

The term “cycloalkyl” as employed herein includes saturated and partially unsaturated cyclic hydrocarbon groups having 3 to 12 carbons, for example 3 to 8 carbons, and as a further example 3 to 6 carbons, wherein the cycloalkyl group additionally is optionally substituted. Examples of cycloalkyl groups include, without limitation, cyclopropyl, cyclobutyl, cyclopentyl, cyclopentenyl, cyclohexyl, cyclohexenyl, cycloheptyl, and cyclooctyl.

The term “heteroalkyl” refers to an alkyl group, as defined hereinabove, wherein one or more carbon atoms in the chain are replaced by a heteroatom selected from the group consisting of O, S, and N.

As used herein, the term “hydroxyalkyl” refers to -alkyl-OH.

The term “dihydroxyalkyl” refers to an alkyl group as defined herein wherein two carbon atoms are each substituted with a hydroxyl group.

The term “alkylaminyl” refers to —NR^{x}-alkyl, wherein R^{x} is hydrogen. In one embodiment, R™ is hydrogen.

The term “dialkylaminyl” refers to —N(R^{y})₂, wherein each R^{y} is C1-C3 alkyl.

The term “alkylaminylalkyl” refers to -alkyl-NR^{x}-alkyl, wherein R^{x} is hydrogen. In one embodiment, R^{x} is hydrogen.

The term “dialkylaminylalkyl” refers to -alkyl-N(R^{y})₂, wherein each R^{y} is C1-C4 alkyl, wherein the alkyl of the -alkyl-N(R^{y})₂ may be optionally substituted with hydroxy or hydroxyalkyl.

An “aryl” group is a C₆-C₁₄ aromatic moiety comprising one to three aromatic rings, which is optionally substituted. As one embodiment, the aryl group is a C₆-C₁₀ aryl group. Examples of aryl groups include, without limitation, phenyl, naphthyl, anthracenyl, fluorenyl, and dihydrobenzofuranyl.

An “aralkyl” or “arylalkyl” group comprises an aryl group covalently linked to an alkyl group, either of which may independently be optionally substituted or unsubstituted. An example of an aralkyl group is (C₁-C₆)alkyl(C₆-C₁₀) aryl, including, without limitation, benzyl, phenethyl, and naphthylmethyl. An example of a substituted aralkyl is wherein the alkyl group is substituted with hydroxyalkyl.

A “heterocyclyl” or “heterocyclic” group is a ring structure having from about 3 to about 12 atoms, for example 4 to 8 atoms, wherein one or more atoms are selected from the group consisting of N, O, and S, the remainder of the ring atoms being carbon. The heterocyclyl may be a monocyclic, a bicyclic, a spirocyclic or a bridged ring system. The heterocyclic group is optionally substituted with R⁷ on carbon or nitrogen at one or more positions, wherein R⁷ is as defined for Formula I. The heterocyclic group is also independently optionally substituted on nitrogen with alkyl, aryl, aralkyl, alkylcarbonyl, alkylsulfonyl, arylcarbonyl, arylsulfonyl, alkoxycarbonyl, aralkoxycarbonyl, or on sulfur with oxo or lower alkyl. Examples of heterocyclic groups include, without limitation, epoxy, azetidinyl, aziridinyl, tetrahydrofuranyl, tetrahydropyranyl, pyrrolidinyl, pyrrolidinonyl, piperidinyl, piperazinyl, imidazolidinyl, thiazolidinyl, dithianyl, trithianyl, dioxolanyl, oxazolidinyl, oxazolidinonyl, decahydroquinolinyl, piperidonyl, 4-piperidinonyl, thiomorpholinyl, thiomorpholinyl 1,1 dioxide, morpholinyl, oxazepanyl, azabicyclohexanes, azabicycloheptanes and oxa azabiocycloheptanes. Specifically excluded from the scope of this term are compounds having adjacent annular O and/or S atoms.

The term “heterocyclylalkyl” refers to a heterocyclyl group as defined herein linked to the remaining portion of the molecule via an alkyl linker, wherein the alkyl linker of the heterocyclylalkyl may be optionally substituted with hydroxy or hydroxyalkyl.

As used herein, the term “heteroaryl” refers to groups having 5 to 14 ring atoms, preferably 5, 6, 9, or 10 ring atoms; having 6, 10, or 14π electrons shared in a cyclic array; and having, in addition to carbon atoms, from one to three heteroatoms per ring selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

A “heteroarylalkyl” group comprises a heteroaryl group covalently linked to an alkyl group, wherein the radical is on the alkyl group, either of which is independently optionally substituted or unsubstituted. Examples of heteroarylalkyl groups include a heteroaryl group having 5, 6, 9, or 10 ring atoms bonded to a C₁-C₆ alkyl group. Examples of heteroaralkyl groups include pyridylmethyl, pyridylethyl, pyrrolylmethyl, pyrrolylethyl, imidazolylmethyl, imidazolylethyl, thiazolylmethyl, thiazolylethyl, benzimidazolylmethyl, benzimidazolylethyl quinazolinylmethyl, quinolinylmethyl, quinolinylethyl, benzofuranylmethyl, indolinylethyl isoquinolinylmethyl, isoinodylmethyl, cinnolinylmethyl, and benzothiophenylethyl. Specifically excluded from the scope of this term are compounds having adjacent annular O and/or S atoms.

As used herein, “an effective amount” of a compound is an amount that is sufficient to negatively modulate or inhibit the activity of the desired target, i.e., PARP or KRas G12C. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of PARP or KRas G12C. Such amount may be administered as a single dosage or may be administered according to a regimen, whereby it is effective.

As used herein, a “therapeutically effective amount of a combination” of two compounds is an amount that together synergistically increases the activity of the combination in comparison to the therapeutically effective amount of each compound in the combination, i.e., more than merely additive. Alternatively, in vivo, the therapeutically effective amount of the combination of a PARP inhibitor, or a pharmaceutically acceptable salt or a pharmaceutical composition thereof, and a KRas G12C inhibitor compound of Formula (I), Formula I-A, or Formula I-B, or a pharmaceutically acceptable salts or a pharmaceutical composition thereof, results in an increased duration of overall survival (“OS”) in subjects relative to treatment with only the KRas G12 inhibitor. In one embodiment, the therapeutically effective amount of the combination of a PARP inhibitor, or a pharmaceutically acceptable salt or a pharmaceutical composition thereof, and a KRas G12C inhibitor compound of Formula (I), Formula I-A, or Formula I-B, or a pharmaceutically acceptable salt or a pharmaceutical composition thereof, results in an increased duration of progression-free survival (“PFS”) in subjects relative to treatment with only the KRas G12 inhibitor. In one embodiment, the therapeutically effective amount of the combination of a PARP inhibitor, or a pharmaceutically acceptable salt or a pharmaceutical composition thereof, and a KRas G12C inhibitor compound of Formula (I), Formula I-A, or Formula I-B, or a pharmaceutically acceptable salt or pharmaceutical compositions thereof, results in increased tumor regression in subjects relative to treatment with only the KRas G12C inhibitor. In one embodiment, the therapeutically effective amount of the combination of a PARP inhibitor, or a pharmaceutically acceptable salt or a pharmaceutical composition thereof, and a KRas G12C inhibitor compound of Formula (I), Formula I-A, or Formula I-B, or a pharmaceutically acceptable salt or a pharmaceutical composition thereof, results in increased tumor growth inhibition in subjects relative to treatment with only the KRas G12C inhibitor. In one embodiment, the therapeutically effective amount of the combination of a PARP inhibitor, or a pharmaceutically acceptable salt or a pharmaceutical composition thereof, and a KRas G12C inhibitor compound of Formula (I), Formula I-A, or Formula I-B, or a pharmaceutically acceptable salt or a pharmaceutical composition thereof, results in an improvement in the duration of stable disease in subjects compared to treatment with only the KRas G12 inhibitor. The amount of each compound in the combination may be the same or different than the therapeutically effective amount of each compound when administered alone as a monotherapy as long as the combination is synergistic. Such amounts may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

As used herein, the term “about” when used to modify a numerically defined parameter (e.g., the dose of a KRAS inhibitor or a PARP inhibitor or a pharmaceutically acceptable salt thereof, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

### Inhibitor Compounds

In one aspect of the invention, provided herein are methods of treating cancer in a subject in need thereof, comprising administering to the subject a therapeutically effective amount of a combination of a PARP inhibitor, or a pharmaceutically acceptable salt or a pharmaceutical composition thereof, and a KRAS G12C inhibitor of Formula (I), Formula I-A or Formula I-B, or a pharmaceutically acceptable salt or a pharmaceutical composition thereof.

Methods for manufacturing PARP inhibitors, or pharmaceutically acceptable salts or pharmaceutical compositions thereof are well known to those skilled in the art and PARP inhibitors may be obtained from a wide-variety of commercial suppliers, in forms suitable for both research or human use. In addition, suitable PARP inhibitors for use in the compositions and methods disclosed herein, methods for preparing such inhibitors, and related uses and methods are disclosed in U.S. patents: U.S. Pat. Nos. 7,151,102; 7,449,464; 7,981,889; 8,071,579; 8,143,241; 8,247,416; 8,475,842; 8,859,562; 8,912,187; 9,169,235; 9,566,276; 8,012,976; 8,420,650; 8,735,392; 9,820,985; 10,189,837; 6,495,541; 7,351,701; 7,531,530; 8,754,072; 9,045,487; 9,861,638; 9,987,285; 10,130,636; 10,278,974; 8,071,623; 8,436,185; 11,091,459; 7,550,603 and others.

### 1. KRas G12C Inhibitors

In one embodiment, the KRas G12C inhibitors used in the methods are compounds of Formula (I):

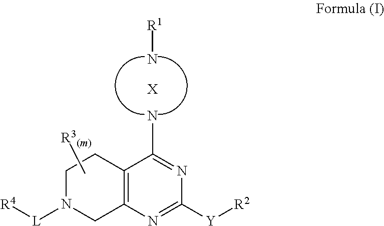

or a pharmaceutically acceptable salt thereof, wherein:
X is a 4-12 membered saturated or partially saturated monocyclic, bridged or spirocyclic ring, wherein the saturated or partially saturated monocyclic ring is optionally substituted with one or more R⁸;
Y is a bond, O, S or NR⁵;
R¹ is —C(O)C(R^{A}) [symbol] C(R^{B})^{p} or —SO₂C(R^{A}) [symbol] C(R^{B})ₚ;
R² is hydrogen, alkyl, hydroxyalkyl, dihydroxyalkyl, alkylaminylalkyl, dialkylaminylalkyl, —Z—NR⁵R¹⁰, heterocyclyl, heterocyclylalkyl, aryl, heteroaryl, or heteroarylalkyl, wherein each of the Z, heterocyclyl, heterocyclylalkyl, aryl, heteroaryl, and heteroarylalkyl may be optionally substituted with one or more R⁹;
Z is C1-C4 alkylene;
each R³ is independently C1-C3 alkyl, oxo, or haloalkyl;
L is a bond, —C(O)—, or C1-C3 alkylene;
R⁴ is hydrogen, cycloalkyl, heterocyclyl, aryl, aralkyl or heteroaryl, wherein each of the cycloalkyl, heterocyclyl, aryl, aralkyl and heteroaryl may be optionally substituted with one or more R⁶ or R⁷;
each R⁵ is independently hydrogen or C1-C3 alkyl;
R⁶ is cycloalkyl, heterocyclyl, heterocyclylalkyl, aryl, or heteroaryl, wherein each of the cycloalkyl, heterocyclyl, aryl, or heteroaryl may be optionally substituted with one or more R⁷;
each R⁷ is independently halogen, hydroxyl, C1-C6 alkyl, cycloalkyl, alkoxy, haloalkyl, amino, cyano, heteroalkyl, hydroxyalkyl or Q-haloalkyl, wherein Q is O or S;
R⁸ is oxo, C1-C3 alkyl, C2-C4 alkynyl, heteroalkyl, cyano, —C(O)OR⁵, —C(O)N(R⁵)₂, —N(R⁵)₂, wherein the C1-C3 alkyl may be optionally substituted with cyano, halogen, —OR⁵, —N(R⁵)₂, or heteroaryl;
each R⁹ is independently hydrogen, oxo, acyl, hydroxyl, hydroxyalkyl, cyano, halogen, C1-C6 alkyl, aralkyl, haloalkyl, heteroalkyl, cycloalkyl, heterocyclylalkyl, alkoxy, dialkylaminyl, dialkylamidoalkyl, or dialkylaminylalkyl, wherein the C1-C6 alkyl may be optionally substituted with cycloalkyl;
each R¹⁰ is independently hydrogen, acyl, C1-C3 alkyl, heteroalkyl or hydroxyalkyl; R¹¹ is haloalkyl;
R^{A} is absent, hydrogen, deuterium, cyano, halogen, C1-C-3 alkyl, haloalkyl, heteroalkyl, —C(O)N(R⁵)₂, or hydroxyalkyl;
each R^{B} is independently hydrogen, deuterium, cyano, C1-C3 alkyl, hydroxyalkyl, heteroalkyl, C1-C3 alkoxy, halogen, haloalkyl, —ZNR⁵R¹¹, —C(O)N(R⁵)₂, —NHC(O)C1-C3 alkyl, —CH₂NHC(O)C1-C3 alkyl, heteroaryl, heteroarylalkyl, dialkylaminylalkyl, or heterocyclylalkyl wherein the heterocyclyl portion is substituted with one or more substituents independently selected from halogen, hydroxyl, alkoxy and C1-C3 alkyl, wherein the heteroaryl or the heteroaryl portion of the heteroarylalkyl is optionally substituted with one or more R⁷;
m is zero or an integer between 1 and 2;
p is one or two; and wherein,
when [symbol] is a triple bond then R^{A} is absent, R^{B} is present and p equals one;
or when [symbol] is a double bond then R^{A} is present, R^{B} is present and p equals two, or R^{A}, R^{B} and the carbon atoms to which they are attached form a 5-8 membered partially saturated cycloalkyl optionally substituted with one or more R⁷.

In one embodiment, KRas G12C inhibitors used in the methods herein includes compounds having the Formula I-A:

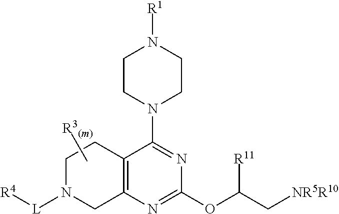

or a pharmaceutically acceptable salt thereof, wherein R¹, R³, R⁴, R⁵, R¹⁰, L and m are as defined for Formula I, R¹¹ is hydrogen, methyl or hydroxyalkyl, and the piperidinyl ring is optionally substituted with R⁸ wherein R⁸ is as defined for Formula I.

In one embodiment, KRas G12C inhibitors used in the methods herein include compounds having the Formula I-B:

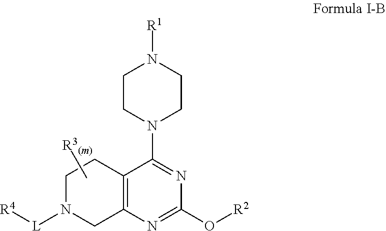

or a pharmaceutically acceptable salt thereof, wherein R¹, R³, R⁴, R⁹, R¹¹, L and m are as defined for Formula I.

Nonlimiting examples of KRas G12C inhibitor compounds of Formula (I), Formula I-A and Formula I-B useful in the methods disclosed herein are selected from the group consisting of Example Nos 1-678 of WO2019/099524 and Example Nos 1-228 of WO2020/101736, including the representative structures:

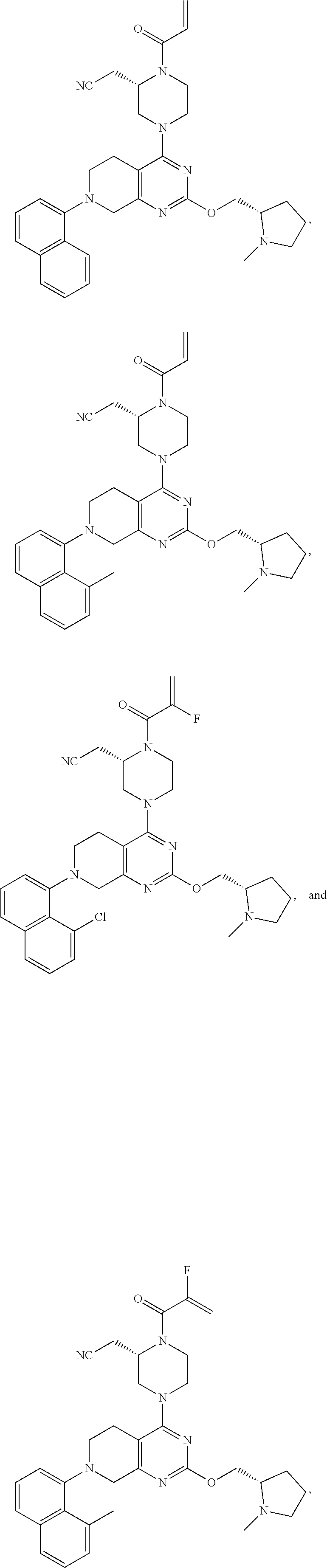 [SMILES: C=CC(=O)N1CCN(c2nc(OC[C@@H]3CCCN3C)nc3c2CCN(c2cccc4ccccc24)C3)C[C@@H]1CC#N ; C=CC(=O)N1CCN(c2nc(OC[C@@H]3CCCN3C)nc3c2CCN(c2cccc4cccc(C)c24)C3)C[C@@H]1CC#N ; C=C(F)C(=O)N1CCN(c2nc(OC[C@@H]3CCCN3C)nc3c2CCN(c2cccc4cccc(Cl)c24)C3)C[C@@H]1CC#N ; C=C(F)C(=O)N1CCN(c2nc(OC[C@@H]3CCCN3C)nc3c2CCN(c2cccc4cccc(C)c24)C3)C[C@@H]1CC#N]

and pharmaceutically acceptable salts thereof.

In one embodiment, the KRas G12C inhibitor is:

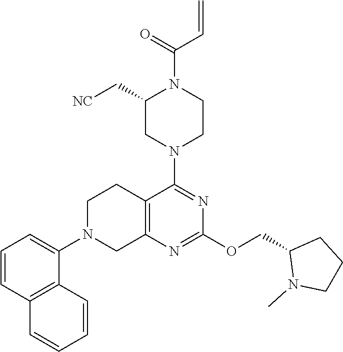 [SMILES: C=CC(=O)N1CCN(c2nc(OC[C@@H]3CCCN3C)nc3c2CCN(c2cccc4ccccc24)C3)C[C@@H]1CC#N]

(also referred to as Example 234 of WO2019/099524) or a pharmaceutically acceptable salt thereof.

In one embodiment, the KRas G12C inhibitor is:

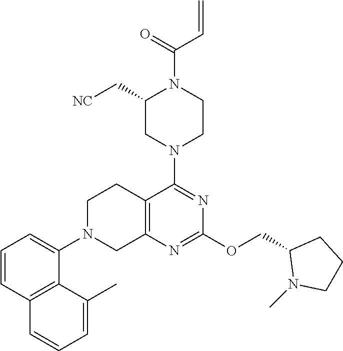 [SMILES: C=CC(=O)N1CCN(c2nc(OC[C@@H]3CCCN3C)nc3c2CCN(c2cccc4cccc(C)c24)C3)C[C@@H]1CC#N]

(also referred to as Example 359 of WO2019/099524) or a pharmaceutically acceptable salt thereof.

In one embodiment, the KRas G12C inhibitor is:

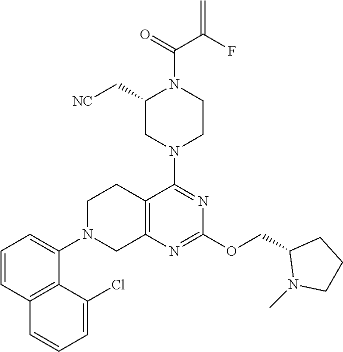 [SMILES: C=C(F)C(=O)N1CCN(c2nc(OC[C@@H]3CCCN3C)nc3c2CCN(c2cccc4cccc(Cl)c24)C3)C[C@@H]1CC#N]

(also referred to as Example 478 of WO2019/099524, MRTX-849 and/or adagrasib) or a pharmaceutically acceptable salt thereof.

In one embodiment, the KRas G12C inhibitor is:

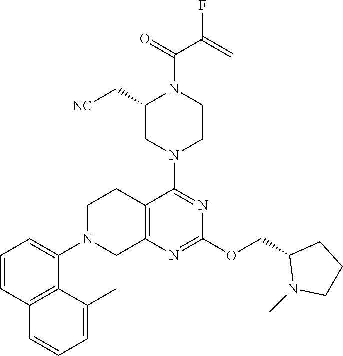 [SMILES: C=C(F)C(=O)N1CCN(c2nc(OC[C@@H]3CCCN3C)nc3c2CCN(c2cccc4cccc(C)c24)C3)C[C@@H]1CC#N]

(also referred to as Example 507 of WO2019/099524) or a pharmaceutically acceptable salt thereof.

The KRas G12C inhibitors used in the methods of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

In one embodiment, the KRas G12C inhibitor compounds of Formula I, Formula I-A, Formula I-B or the aforementioned chemical species used in the methods include trifluoroacetic acid salts of the above compounds.

Methods for manufacturing the KRas G12C inhibitors disclosed herein are known. For example, commonly owned published international PCT application numbers WO2017201161 and WO2019099524, and published US patent application publication number US20180072723 and US20190270743 describe general reaction schemes for preparing compounds of Formula I, Formula I-A, or Formula I-B, or a pharmaceutically acceptable salt or a pharmaceutical composition thereof, and also provide detailed synthetic routes for the preparation of each KRas G12C inhibitor disclosed herein.

The PARP inhibitors and the KRas G12C compounds of Formula (I), Formula I-A, or Formula I-B, or a pharmaceutically acceptable salt or a pharmaceutical composition thereof, may be formulated into pharmaceutical compositions.

### Pharmaceutical Compositions

In another aspect, the invention provides pharmaceutical compositions comprising a PARP inhibitor, or a pharmaceutically acceptable salt thereof and KRas G12C inhibitor, or a pharmaceutically acceptable salt thereof, according to the invention and a pharmaceutically acceptable carrier, excipient, or diluent that may be used in the methods disclosed herein. The PARP inhibitor, or a pharmaceutically acceptable salt thereof and KRas G12C inhibitor, or a pharmaceutically acceptable salt thereof, may be formulated by any method well known in the art and may be prepared for administration by any route, including, without limitation, parenteral, oral, sublingual, transdermal, topical, intranasal, intratracheal, or intrarectal. In certain embodiments, PARP inhibitor, or a pharmaceutically acceptable salt thereof and KRas G12C inhibitor, or a pharmaceutically acceptable salt thereof, are administered intravenously in a hospital setting. In one embodiment, administration may be by the oral route.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18th Edition, ed. A. Gennaro, Mack Publishing Co., Easton, Pa., 1990.

As used herein, the term pharmaceutically acceptable salt refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising a PARP inhibitor, or a pharmaceutically acceptable salt thereof and a KRas G12C inhibitor, or a pharmaceutically acceptable salt thereof may be used in the methods of use described herein.

### Co-Administration

The PARP inhibitor, or a pharmaceutically acceptable salt or a pharmaceutical composition thereof, and the KRas G12C inhibitor, or a pharmaceutically acceptable salt or a pharmaceutical composition thereof, can be independently formulated into separate or individual dosage forms which can be co-administered one after the other. Another option is that if the route of administration is the same (e.g. oral) two active compounds can be formulated into a single form for co-administration, both methods of co-administration, however, being part of the same therapeutic treatment or regimen.

The pharmaceutical compositions comprising a PARP inhibitor, or a pharmaceutically acceptable salt thereof and/or a KRas G12C inhibitor, or a pharmaceutically acceptable salt thereof for use in the methods may be for simultaneous, separate or sequential use. In one embodiment, the PARP inhibitor, or a pharmaceutically acceptable salt or a pharmaceutical composition thereof, is administered prior to administration of the KRas G12C inhibitor compound of Formula (I), Formula I-A, Formula I-B, or selected from the aforementioned chemical species, or a pharmaceutically acceptable salt or a pharmaceutical composition thereof. In another embodiment, the PARP inhibitor, or a pharmaceutically acceptable salt or a pharmaceutical composition thereof, is administered after administration of the KRas G12C inhibitor compound of Formula (I), Formula I-A, Formula I-B or selected from the aforementioned chemical species, or a pharmaceutically acceptable salt or a pharmaceutical composition thereof. In another embodiment, the PARP inhibitor, or a pharmaceutically acceptable salt or a pharmaceutical composition thereof, is administered at about the same time as administration of the KRas G12C inhibitor compound of Formula (I), Formula I-A, Formula I-B, or selected from the aforementioned chemical species, or a pharmaceutically acceptable salt or a pharmaceutical composition thereof.

Separate administration of each inhibitor, at different times and by different routes, in some cases would be advantageous. Thus, the components in the combination i.e. the KRas G12C inhibitor compound of Formula (I), Formula I-A or Formula I-B, or a pharmaceutically acceptable salt or a pharmaceutical composition thereof, and the PARP inhibitor, or a pharmaceutically acceptable salt or a pharmaceutical composition thereof, need not be necessarily administered at essentially the same time or in any order.

Oncology drugs are typically administered at the maximum tolerated dose (“MTD”), which is the highest dose of drug that does not cause unacceptable side effects. In one embodiment, the KRas G12C inhibitor, or a pharmaceutically acceptable salt or a pharmaceutical composition thereof, and the PARP inhibitor, or a pharmaceutically acceptable salt or a pharmaceutical composition thereof, are each dosed at their respective MTDs. In one embodiment, the KRas G12C inhibitor, or a pharmaceutically acceptable salt or a pharmaceutical composition thereof, is dosed at its MTD and the PARP inhibitor, or a pharmaceutically acceptable salt or a pharmaceutical composition thereof, is dosed in an amount less than its MTD. In one embodiment, the KRas G12C inhibitor, or a pharmaceutically acceptable salt or a pharmaceutical composition thereof, is dosed at an amount less than its MTD and the PARP inhibitor, or a pharmaceutically acceptable salt or a pharmaceutical composition thereof, is dosed at its MTD. In one embodiment, the KRas G12C inhibitor, or a pharmaceutically acceptable salt or a pharmaceutical composition thereof, and the PARP inhibitor, or a pharmaceutically acceptable salt or a pharmaceutical composition thereof, are each dosed at less than their respective MTDs. The administration can be so timed that the peak pharmacokinetic effect of one compound coincides with the peak pharmacokinetic effect of the other.

In one embodiment, a single dose of KRas G12C inhibitor compound of Formula (I), Formula I-A, Formula I-B or selected from the aforementioned chemical species, or a pharmaceutically acceptable salt or a pharmaceutical composition thereof, is administered per day (i.e., in about 24 hour intervals) (i.e., QD). In another embodiment, two doses of the KRas G12C inhibitor compound of Formula (I), Formula I-A, Formula I-B or selected from the aforementioned chemical species, or a pharmaceutically acceptable salt or a pharmaceutical composition thereof, are administered per day (i.e., BID). In another embodiment, three doses of the KRas G12C inhibitor compound of Formula (I), Formula I-A, Formula I-B or selected from the aforementioned chemical species, or a pharmaceutically acceptable salt or a pharmaceutical composition thereof are administered per day (i.e., TID).

In one embodiment, the PARP inhibitor, or a pharmaceutically acceptable salt or a pharmaceutical composition thereof, is administered QD. In another embodiment, the PARP inhibitor, or a pharmaceutically acceptable salt or a pharmaceutical composition thereof, is administered BID. In another embodiment, the PARP inhibitor, or a pharmaceutically acceptable salt or a pharmaceutical composition thereof, is administered TID.

In one embodiment, a single dose of KRas G12C inhibitor compound of Formula (I), Formula I-A or Formula I-B, or a pharmaceutically acceptable salt or a pharmaceutical composition thereof, and PARP inhibitor, or a pharmaceutically acceptable salt or a pharmaceutical composition thereof, are each administered once daily.

Examples of PARP inhibitors suitable for the provided compositions and methods include, but are not limited to olaparib, rucaparib, niraparib, talazoparib, and veliparib. Other examples of PARP inhibitors suitable for the provided compositions and methods include, but are not limited to: RBN-2397, I-1, KMR-206, RP14042 and JAB-26766.

### Combination Therapies

In one aspect of the invention, provided herein are methods of treating cancer in a subject in need thereof, comprising administering to the subject a therapeutically effective amount of a combination of a PARP inhibitor, or a pharmaceutically acceptable salt or a pharmaceutical composition thereof, and a KRAS G12C inhibitor of Formula (I), Formula I-A, Formula I-B or selected from the aforementioned chemical species (i.e.,

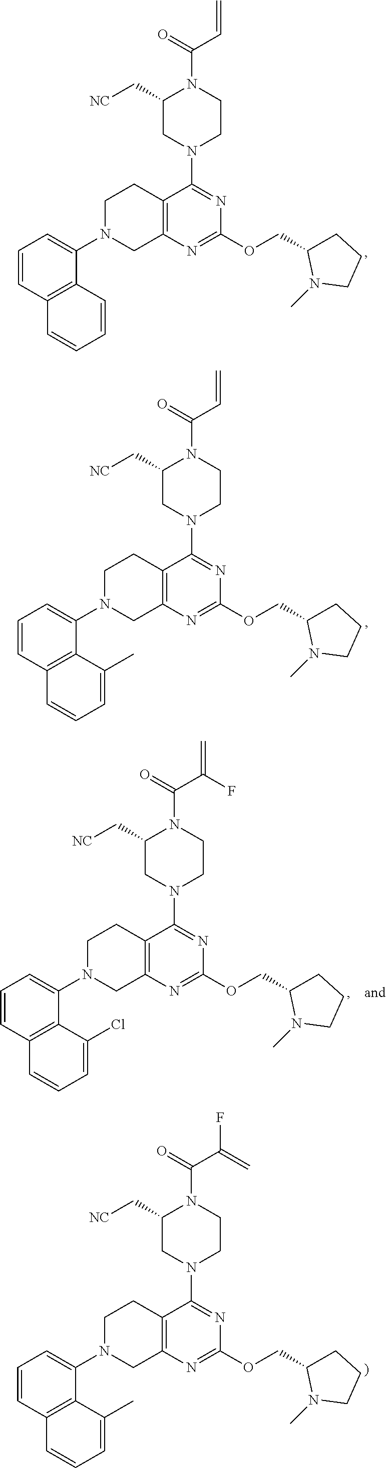 [SMILES: C=CC(=O)N1CCN(c2nc(OC[C@@H]3CCCN3C)nc3c2CCN(c2cccc4ccccc24)C3)C[C@@H]1CC#N ; C=CC(=O)N1CCN(c2nc(OC[C@@H]3CCCN3C)nc3c2CCN(c2cccc4cccc(C)c24)C3)C[C@@H]1CC#N ; C=C(F)C(=O)N1CCN(c2nc(OC[C@@H]3CCCN3C)nc3c2CCN(c2cccc4cccc(Cl)c24)C3)C[C@@H]1CC#N ; C=C(F)C(=O)N1CCN(c2nc(OC[C@@H]3CCCN3C)nc3c2CCN(c2cccc4cccc(C)c24)C3)C[C@@H]1CC#N]

or a pharmaceutically acceptable salt or a pharmaceutical composition thereof. In one embodiment, the cancer is a KRas G12C-associated cancer. In one embodiment, the KRas G12C-associated cancer is lung cancer.

In yet another aspect, the invention provides for methods for increasing the sensitivity of a cancer cell to a KRas G12C inhibitor, comprising contacting the cancer cell with an effective amount of a combination of a KRas G12C inhibitor compound of Formula (I), Formula I-A, Formula I-B or selected from the aforementioned chemical species, or a pharmaceutically acceptable salt or a pharmaceutical composition thereof, and a PARP inhibitor, or a pharmaceutically acceptable salt or a pharmaceutical composition thereof, wherein the PARP inhibitor synergistically increases the sensitivity of the cancer cell to the KRas G12C inhibitor. In one embodiment, the contacting is in vitro. In one embodiment, the contacting is in vivo.

In one embodiment, the combination therapy comprises a combination of a compound having the formula:

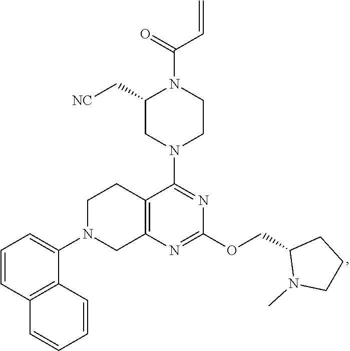 [SMILES: C=CC(=O)N1CCN(c2nc(OC[C@@H]3CCCN3C)nc3c2CCN(c2cccc4ccccc24)C3)C[C@@H]1CC#N]

or a pharmaceutically acceptable salt thereof, and PARP inhibitor. In one aspect of this embodiment, the PARP inhibitor is olaparib. In one aspect of this embodiment, the PARP inhibitor is rucaparib. In one aspect of this embodiment, the PARP inhibitor is niraparib. In one aspect of this embodiment, the PARP inhibitor is talazoparib. In one aspect of this embodiment, the PARP inhibitor is veliparib.

In one embodiment, the combination therapy comprises a combination of a compound having the formula:

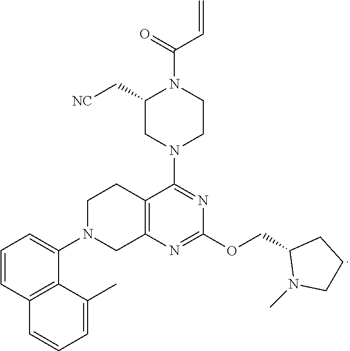 [SMILES: C=CC(=O)N1CCN(c2nc(OC[C@@H]3CCCN3C)nc3c2CCN(c2cccc4cccc(C)c24)C3)C[C@@H]1CC#N]

or a pharmaceutically acceptable salt thereof, and a PARP inhibitor.

In one aspect of this embodiment, the PARP inhibitor is olaparib. In one aspect of this embodiment, the PARP inhibitor is rucaparib. In one aspect of this embodiment, the PARP inhibitor is niraparib. In one aspect of this embodiment, the PARP inhibitor is talazoparib. In one aspect of this embodiment, the PARP inhibitor is veliparib.

In one embodiment, the combination therapy comprises a combination of a compound having the formula:

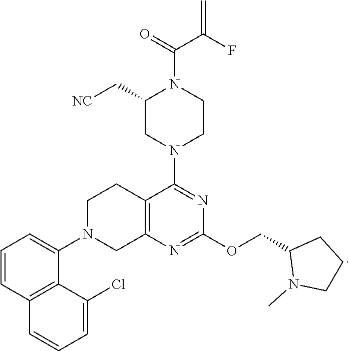

or a pharmaceutically acceptable salt thereof, and a PARP inhibitor.

In one aspect of this embodiment, the PARP inhibitor is olaparib. In one aspect of this embodiment, the PARP inhibitor is rucaparib. In one aspect of this embodiment, the PARP inhibitor is niraparib. In one aspect of this embodiment, the PARP inhibitor is talazoparib. In one aspect of this embodiment, the PARP inhibitor is veliparib.

In one embodiment, the combination therapy comprises a combination of a compound having the formula:

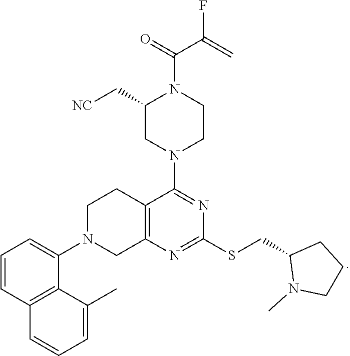 [SMILES: C=C(F)C(=O)N1CCN(c2nc(SC[C@@H]3CCCN3C)nc3c2CCN(c2cccc4cccc(C)c24)C3)C[C@@H]1CC#N]

or a pharmaceutically acceptable salt thereof, and a PARP inhibitor.

In one aspect of this embodiment, the PARP inhibitor is olaparib. In one aspect of this embodiment, the PARP inhibitor is rucaparib. In one aspect of this embodiment, the PARP inhibitor is niraparib. In one aspect of this embodiment, the PARP inhibitor is talazoparib. In one aspect of this embodiment, the PARP inhibitor is veliparib.

As used herein, the term “contacting” refers to the bringing together of indicated moieties in an in vitro system or an in vivo system. For example, “contacting” a cancer cell includes the administration of a combination provided herein to an individual or subject, such as a human, having KRas G12C, as well as, for example, introducing a combination provided herein into a sample containing a cellular or purified preparation containing KRas G12C.

By negatively modulating the activity of KRas G12C, the methods described herein are designed to inhibit undesired cellular proliferation resulting from enhanced KRas G12C activity within the cell. The degree of covalent modification of KRas G12C may be monitored in vitro using well known methods, including those described in published international PCT application numbers WO2017201161, WO2019099524 and WO2020101736. In addition, the inhibitory activity of combination in cells may be monitored, for example, by measuring the inhibition of KRas G12C activity of the amount of phosphorylated ERK to assess the effectiveness of treatment and dosages may be adjusted accordingly by the attending medical practitioner.

The compositions and methods provided herein may be used for the treatment of a KRas G12C-associated cancer in a subject in need thereof, comprising administering to said subject a therapeutically effective amount of a combination of a PARP inhibitor, or a pharmaceutically acceptable salt or a pharmaceutical composition thereof, and a KRas G12C inhibitor compound of Formula (I), Formula I-A, or Formula I-B, or a pharmaceutically acceptable salt or a pharmaceutical composition thereof, wherein the PARP inhibitor synergistically increases the sensitivity of the KRas G12C-associated cancer to the KRas G12C inhibitor. In one embodiment, the KRas G12C-associated cancer is lung cancer.

In one embodiment, the therapeutically effective amount of the combination of a PARP inhibitor, a pharmaceutically acceptable salt or a pharmaceutical composition thereof, and a KRas G12C inhibitor compound of Formula (I), Formula I-A, or Formula I-B, or a pharmaceutically acceptable salt or a pharmaceutical composition thereof, results in an increased duration of overall survival (“OS”) in subjects relative to treatment with only the KRas G12C inhibitor. In one embodiment, the therapeutically effective amount of the combination of a PARP inhibitor, or a pharmaceutically acceptable salt or a pharmaceutical composition thereof, and a KRas G12C inhibitor compound of Formula (I), Formula I-A, or Formula I-B, a pharmaceutically acceptable salt or a pharmaceutical composition thereof, results in an increased duration of progression-free survival (“PFS”) in subjects relative to treatment with only the KRas G12C inhibitor. In one embodiment, the therapeutically effective amount of the combination of a PARP inhibitor, or a pharmaceutically acceptable salt or a pharmaceutical composition thereof, and a KRas G12C inhibitor compound of Formula (I), Formula I-A, or Formula I-B, or a pharmaceutically acceptable salt or a pharmaceutical composition thereof, results in increased tumor regression in subjects relative to treatment with only the KRas G12C inhibitor. In one embodiment, the therapeutically effective amount of the combination of a PARP inhibitor, or a pharmaceutically acceptable salt or a pharmaceutical composition thereof, and a KRas G12C inhibitor compound of Formula (I), Formula I-A, or Formula I-B, or a pharmaceutically acceptable salt or a pharmaceutical composition thereof, results in increased tumor growth inhibition in subjects relative to treatment with only the KRas G12C inhibitor. In one embodiment, the therapeutically effective amount of the combination of a PARP inhibitor, or a pharmaceutically acceptable salt or a pharmaceutical composition thereof, and a KRas G12C inhibitor compound of Formula (I), Formula I-A, or Formula I-B, or a pharmaceutically acceptable salt or a pharmaceutical composition thereof, results in an improvement in the duration of stable disease in subjects compared to treatment with only the KRas G12C inhibitor. In one embodiment, the KRas G12C inhibitor is a compound selected from compound Nos. 1-678 (as numbered in WO2019099524), or a pharmaceutically acceptable salt thereof (e.g., Example No. 234, 359, 478 or 507 or a pharmaceutically acceptable salt thereof). In one embodiment, the PARP inhibitor is selected from olaparib, rucaparib, niraparib, talazoparib, and veliparib. In one embodiment, the therapeutic combination comprises therapeutically effective amounts of Example No. 234 and olaparib. In one embodiment, the therapeutic combination comprises therapeutically effective amounts of Example No. 234 and rucaparib. In one embodiment, the therapeutic combination comprises therapeutically effective amounts of Example No. 234 and niraparib. In one embodiment, the therapeutic combination comprises therapeutically effective amounts of Example No. 234 and talazoparib. In one embodiment, the therapeutic combination comprises therapeutically effective amounts of Example No. 234 and veliparib. In one embodiment, the therapeutic combination comprises therapeutically effective amounts of Example No. 359 and olaparib. In one embodiment, the therapeutic combination comprises therapeutically effective amounts of Example No. 359 and rucaparib. In one embodiment, the therapeutic combination comprises therapeutically effective amounts of Example No. 359 and niraparib. In one embodiment, the therapeutic combination comprises therapeutically effective amounts of Example No. 359 and talazoparib. In one embodiment, the therapeutic combination comprises therapeutically effective amounts of Example No. 359 and veliparib. In one embodiment, the therapeutic combination comprises therapeutically effective amounts of Example No. 478 and olaparib. In one embodiment, the therapeutic combination comprises therapeutically effective amounts of Example No. 478 and rucaparib. In one embodiment, the therapeutic combination comprises therapeutically effective amounts of Example No. 478 and niraparib. In one embodiment, the therapeutic combination comprises therapeutically effective amounts of Example No. 478 and talazoparib. In one embodiment, the therapeutic combination comprises therapeutically effective amounts of Example No. 478 and veliparib. In one embodiment, the therapeutic combination comprises therapeutically effective amounts of Example No. 507 and olaparib. In one embodiment, the therapeutic combination comprises therapeutically effective amounts of Example No. 507 and rucaparib. In one embodiment, the therapeutic combination comprises therapeutically effective amounts of Example No. 507 and niraparib. In one embodiment, the therapeutic combination comprises therapeutically effective amounts of Example No. 507 and talazoparib. In one embodiment, the therapeutic combination comprises therapeutically effective amounts of Example No. 507 and veliparib.

In another embodiment, the PARP inhibitor, or a pharmaceutically acceptable salt or a pharmaceutical composition thereof, is administered in combination with the KRas G12C inhibitor, or a pharmaceutically acceptable salt or a pharmaceutical composition thereof once disease progression has been observed for KRas G12C monotherapy, in which the combination therapy results in enhanced clinical benefit for the patient by increasing OS, PFS, tumor regression, tumor growth inhibition or the duration of stable disease in the patient.

In one embodiment, the KRas G12C inhibitor is a compound selected from compound Nos. 1-678 (as numbered in WO2019099524), or a pharmaceutically acceptable salt thereof (e.g., Example No. 234, 359, 478 or 507 or a pharmaceutically acceptable salt thereof). In one embodiment, the PARP inhibitor is selected from olaparib, rucaparib, niraparib, talazoparib, and veliparib. In one embodiment, the therapeutic combination comprises therapeutically effective amounts of Example No. 234 and olaparib. In one embodiment, the therapeutic combination comprises therapeutically effective amounts of Example No. 234 and rucaparib. In one embodiment, the therapeutic combination comprises therapeutically effective amounts of Example No. 234 and niraparib. In one embodiment, the therapeutic combination comprises therapeutically effective amounts of Example No. 234 and talazoparib. In one embodiment, the therapeutic combination comprises therapeutically effective amounts of Example No. 234 and veliparib. In one embodiment, the therapeutic combination comprises therapeutically effective amounts of Example No. 359 and olaparib. In one embodiment, the therapeutic combination comprises therapeutically effective amounts of Example No. 359 and rucaparib. In one embodiment, the therapeutic combination comprises therapeutically effective amounts of Example No. 359 and niraparib. In one embodiment, the therapeutic combination comprises therapeutically effective amounts of Example No. 359 and talazoparib. In one embodiment, the therapeutic combination comprises therapeutically effective amounts of Example No. 359 and veliparib. In one embodiment, the therapeutic combination comprises therapeutically effective amounts of Example No. 478 and olaparib. In one embodiment, the therapeutic combination comprises therapeutically effective amounts of Example No. 478 and rucaparib. In one embodiment, the therapeutic combination comprises therapeutically effective amounts of Example No. 478 and niraparib. In one embodiment, the therapeutic combination comprises therapeutically effective amounts of Example No. 478 and talazoparib. In one embodiment, the therapeutic combination comprises therapeutically effective amounts of Example No. 478 and veliparib. In one embodiment, the therapeutic combination comprises therapeutically effective amounts of Example No. 507 and olaparib. In one embodiment, the therapeutic combination comprises therapeutically effective amounts of Example No. 507 and rucaparib. In one embodiment, the therapeutic combination comprises therapeutically effective amounts of Example No. 507 and niraparib. In one embodiment, the therapeutic combination comprises therapeutically effective amounts of Example No. 507 and talazoparib. In one embodiment, the therapeutic combination comprises therapeutically effective amounts of Example No. 507 and veliparib.

The compositions and methods provided herein may be used for the treatment of a wide variety of cancers including tumors such as lung, colorectal, pancreas, prostate, breast, brain, skin, cervical carcinomas, testicular carcinomas, etc. More particularly, cancers that may be treated by the compositions and methods of the invention include, but are not limited to, tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer.

Also provided herein is a method for treating cancer in a subject in need thereof, the method comprising (a) determining that cancer is associated with a KRas G12C mutation (e.g., a KRas G12C-associated cancer) (e.g., as determined using a regulatory agency-approved, e.g., FDA-approved, assay or kit); and (b) administering to the patient a therapeutically effective amount of a combination of a PARP inhibitor, or a pharmaceutically acceptable salt or a pharmaceutical composition thereof and a KRas G12C inhibitor compound of Formula I, Formula I-A, Formula 1-B, or a pharmaceutically acceptable salt or a pharmaceutical composition thereof, wherein the PARP inhibitor synergistically increases the sensitivity of the KRas G12C-associated cancer to the KRas G12C inhibitor. In one embodiment, the KRas G12C inhibitor is a compound selected from compound Nos. 1-678 (as numbered in WO2019099524), or a pharmaceutically acceptable salt thereof (e.g., Example No. 234, 359, 478 or 507 or a pharmaceutically acceptable salt thereof). In one embodiment, the PARP inhibitor is selected from olaparib, rucaparib, niraparib, talazoparib, and veliparib. In one embodiment, the therapeutic combination comprises therapeutically effective amounts of Example No. 234 and olaparib. In one embodiment, the therapeutic combination comprises therapeutically effective amounts of Example No. 234 and rucaparib. In one embodiment, the therapeutic combination comprises therapeutically effective amounts of Example No. 234 and niraparib. In one embodiment, the therapeutic combination comprises therapeutically effective amounts of Example No. 234 and talazoparib. In one embodiment, the therapeutic combination comprises therapeutically effective amounts of Example No. 234 and veliparib. In one embodiment, the therapeutic combination comprises therapeutically effective amounts of Example No. 359 and olaparib. In one embodiment, the therapeutic combination comprises therapeutically effective amounts of Example No. 359 and rucaparib. In one embodiment, the therapeutic combination comprises therapeutically effective amounts of Example No. 359 and niraparib. In one embodiment, the therapeutic combination comprises therapeutically effective amounts of Example No. 359 and talazoparib. In one embodiment, the therapeutic combination comprises therapeutically effective amounts of Example No. 359 and veliparib. In one embodiment, the therapeutic combination comprises therapeutically effective amounts of Example No. 478 and olaparib. In one embodiment, the therapeutic combination comprises therapeutically effective amounts of Example No. 478 and rucaparib. In one embodiment, the therapeutic combination comprises therapeutically effective amounts of Example No. 478 and niraparib. In one embodiment, the therapeutic combination comprises therapeutically effective amounts of Example No. 478 and talazoparib. In one embodiment, the therapeutic combination comprises therapeutically effective amounts of Example No. 478 and veliparib. In one embodiment, the therapeutic combination comprises therapeutically effective amounts of Example No. 507 and olaparib. In one embodiment, the therapeutic combination comprises therapeutically effective amounts of Example No. 507 and rucaparib. In one embodiment, the therapeutic combination comprises therapeutically effective amounts of Example No. 507 and niraparib. In one embodiment, the therapeutic combination comprises therapeutically effective amounts of Example No. 507 and talazoparib. In one embodiment, the therapeutic combination comprises therapeutically effective amounts of Example No. 507 and veliparib.

In one embodiment, a compound of Formula I is administered as a capsule during the period of time. In one embodiment, a tablet or capsule formulation of a compound of Formula I comprises about 10 mg to about 100 mg (e.g., about 10 mg to about 95 mg, about 10 mg to about 90 mg, about 10 mg to about 85 mg, about 10 mg to about 80 mg, about 10 mg to about 75 mg, about 10 mg to about 70 mg, about 10 mg to about 65 mg, about 10 mg to about 60 mg, about 10 mg to about 55 mg, about 10 mg to about 50 mg, about 10 mg to about 45 mg, about 10 mg to about 40 mg, about 10 mg to about 35 mg, about 10 mg to about 30 mg, about 10 mg to about 25 mg, about 10 mg to about 20 mg, about 10 mg to about 15 mg, about 15 mg to about 100 mg, about 15 mg to about 95 mg, about 15 mg to about 90 mg, about 15 mg to about 85 mg, about 15 mg to about 80 mg, about 15 mg to about 75 mg, about 15 mg to about 70 mg, about 15 mg to about 65 mg, about 15 mg to about 60 mg, about 15 mg to about 55 mg, about 15 mg to about 50 mg, about 15 mg to about 45 mg, about 15 mg to about 40 mg, about 15 mg to about 35 mg, about 15 mg to about 30 mg, about 15 mg to about 25 mg, about 15 mg to about 20 mg, about 20 mg to about 100 mg, about 20 mg to about 95 mg, about 20 mg to about 90 mg, about 20 mg to about 85 mg, about 20 mg to about 80 mg, about 20 mg to about 75 mg, about 20 mg to about 70 mg, about 20 mg to about 65 mg, about 20 mg to about 60 mg, about 20 mg to about 55 mg, about 20 mg to about 50 mg, about 20 mg to about 45 mg, about mg to about 40 mg, about 20 mg to about 35 mg, about 20 mg to about 30 mg, about 20 mg to about 25 mg, about 25 mg to about 100 mg, about 25 mg to about 95 mg, about 25 mg to about 90 mg, about 25 mg to about 85 mg, about 25 mg to about 80 mg, about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about mg to about 40 mg, about 25 mg to about 35 mg, about 25 mg to about 30 mg, about 30 mg to about 100 mg, about 30 mg to about 95 mg, about 30 mg to about 90 mg, about 30 mg to about 85 mg, about 30 mg to about 80 mg, about 30 mg to about 75 mg, about 30 mg to about 70 mg, about 30 mg to about 65 mg, about 30 mg to about 60 mg, about 30 mg to about 55 mg, about 30 mg to about 50 mg, about 30 mg to about 45 mg, about 30 mg to about 40 mg, about mg to about 35 mg, about 35 mg to about 100 mg, about 35 mg to about 95 mg, about 35 mg to about 90 mg, about 35 mg to about 85 mg, about 35 mg to about 80 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 35 mg to about 40 mg, about 40 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 40 mg to about 45 mg, about 45 mg to about 100 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 45 mg to about 50 mg, about 50 mg to about 100 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 50 mg to about 55 mg, about 55 mg to about 100 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 55 mg to about 60 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 60 mg to about 65 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 65 mg to about 70 mg, about 70 mg to about 100 mg, about 70 mg to about 95 mg, about 70 mg to about 90 mg, about 70 mg to about 85 mg, about 70 mg to about 80 mg, about 70 mg to about 75 mg, about 75 mg to about 100 mg, about 75 mg to about 95 mg, about 75 mg to about 90 mg, about 75 mg to about 85 mg, about 75 mg to about 80 mg, about 80 mg to about 100 mg, about 80 mg to about 95 mg, about 80 mg to about 90 mg, about 80 mg to about 85 mg, about 85 mg to about 100 mg, about 85 mg to about 95 mg, about 85 mg to about 90 mg, about 90 mg to about 100 mg, about 90 mg to about 95 mg, about 95 mg to about 100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg) of a compound of Formula I (e.g., a compound selected from compound Nos. 1-678 (as numbered in WO2019099524, e.g., compound No. 234, 359, 478 or 507, or pharmaceutically acceptable salts thereof). In one embodiment, a compound of Formula I is orally administered once a day (QD) on a daily basis during a period of time. In one embodiment, a compound of Formula I is orally administered twice a day (BID) on a daily basis during a period of time. In one embodiment, a compound of Formula I is orally administered in the amount of about 20 mg to about 500 mg (e.g., about 20 mg to about 480 mg, about 20 mg to about 460 mg, about 20 mg to about 440 mg, about 20 mg to about 420 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 500 mg, about 40 mg to about 480 mg, about 40 mg to about 460 mg, about 40 mg to about 440 mg, about 40 mg to about 420 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 500 mg, about 60 mg to about 480 mg, about 60 mg to about 460 mg, about 60 mg to about 440 mg, about 60 mg to about 420 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 500 mg, about 80 mg to about 480 mg, about 80 mg to about 460 mg, about 80 mg to about 440 mg, about 80 mg to about 420 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 500 mg, about 100 mg to about 480 mg, about 100 mg to about 460 mg, about 100 mg to about 440 mg, about 100 mg to about 420 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 500 mg, about 120 mg to about 480 mg, about 120 mg to about 460 mg, about 120 mg to about 440 mg, about 120 mg to about 420 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 500 mg, about 140 mg to about 480 mg, about 140 mg to about 460 mg, about 140 mg to about 440 mg, about 140 mg to about 420 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 500 mg, about 160 mg to about 480 mg, about 160 mg to about 460 mg, about 160 mg to about 440 mg, about 160 mg to about 420 mg, about 160 mg to about 400 mg, about 160 mg to about 380 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 500 mg, about 180 mg to about 480 mg, about 180 mg to about 460 mg, about 180 mg to about 440 mg, about 180 mg to about 420 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 500 mg, about 200 mg to about 480 mg, about 200 mg to about 460 mg, about 200 mg to about 440 mg, about 200 mg to about 420 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 500 mg, about 220 mg to about 480 mg, about 220 mg to about 460 mg, about 220 mg to about 440 mg, about 220 mg to about 420 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 500 mg, about 240 mg to about 480 mg, about 240 mg to about 460 mg, about 240 mg to about 440 mg, about 240 mg to about 420 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 500 mg, about 260 mg to about 480 mg, about 260 mg to about 460 mg, about 260 mg to about 440 mg, about 260 mg to about 420 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 500 mg, about 280 mg to about 480 mg, about 280 mg to about 460 mg, about 280 mg to about 440 mg, about 280 mg to about 420 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 500 mg, about 300 mg to about 480 mg, about 300 mg to about 460 mg, about 300 mg to about 440 mg, about 300 mg to about 420 mg, about 300 mg to about 400 mg, about 300 mg to about 380 mg, about 300 mg to about 360 mg, about 300 mg to about 340 mg, about 300 mg to about 320 mg, about 320 mg to about 500 mg, about 320 mg to about 480 mg, about 320 mg to about 460 mg, about 320 mg to about 440 mg, about 320 mg to about 420 mg, about 320 mg to about 400 mg, about 320 mg to about 380 mg, about 320 mg to about 360 mg, about 320 mg to about 340 mg, about 340 mg to about 500 mg, about 340 mg to about 480 mg, about 340 mg to about 460 mg, about 340 mg to about 440 mg, about 340 mg to about 420 mg, about 340 mg to about 400 mg, about 340 mg to about 380 mg, about 340 mg to about 360 mg, about 360 mg to about 500 mg, about 360 mg to about 480 mg, about 360 mg to about 460 mg, about 360 mg to about 440 mg, about 360 mg to about 420 mg, about 360 mg to about 400 mg, about 360 mg to about 380 mg, about 380 mg to about 500 mg, about 380 mg to about 480 mg, about 380 mg to about 460 mg, about 380 mg to about 440 mg, about 380 mg to about 420 mg, about 380 mg to about 400 mg, about 400 mg to about 500 mg, about 400 mg to about 480 mg, about 400 mg to about 460 mg, about 400 mg to about 440 mg, about 400 mg to about 420 mg, about 420 mg to about 500 mg, about 420 mg to about 480 mg, about 420 mg to about 460 mg, about 420 mg to about 440 mg, about 440 mg to about 500 mg, about 440 mg to about 480 mg, about 440 mg to about 460 mg, about 460 mg to about 500 mg, about 460 mg to about 480 mg, about 480 mg to about 500 mg, about 25, about 50, about 75, about 100, about 150, about 200, about 250, about 300, about 350, about 400, about 450, or about 500 mg), during a period of time.

In one embodiment, the combination therapy comprises oral administration of a compound of Formula I once or twice a day on a daily basis (during a period of time), e.g., in an amount of about 10 mg to about 400 mg (e.g., about 10 mg to about 380 mg, about 10 mg to about 360 mg, about 10 mg to about 340 mg, about 10 mg to about 320 mg, about 10 mg to about 300 mg, about 10 mg to about 280 mg, about 10 mg to about 260 mg, about 10 mg to about 240 mg, about 10 mg to about 220 mg, about 10 mg to about 200 mg, about 10 mg to about 180 mg, about 10 mg to about 160 mg, about 10 mg to about 140 mg, about 10 mg to about 120 mg, about 10 mg to about 100 mg, about 10 mg to about 80 mg, about 10 mg to about 60 mg, about 10 mg to about 40 mg, about 10 mg to about 20 mg, about 20 mg to about 400 mg, about 20 mg to about 380 mg, about 20 mg to about 360 mg, about 20 mg to about 340 mg, about 20 mg to about 320 mg, about 20 mg to about 300 mg, about 20 mg to about 280 mg, about 20 mg to about 260 mg, about 20 mg to about 240 mg, about 20 mg to about 220 mg, about 20 mg to about 200 mg, about 20 mg to about 180 mg, about 20 mg to about 160 mg, about 20 mg to about 140 mg, about 20 mg to about 120 mg, about 20 mg to about 100 mg, about 20 mg to about 80 mg, about 20 mg to about 60 mg, about 20 mg to about 40 mg, about 40 mg to about 400 mg, about 40 mg to about 380 mg, about 40 mg to about 360 mg, about 40 mg to about 340 mg, about 40 mg to about 320 mg, about 40 mg to about 300 mg, about 40 mg to about 280 mg, about 40 mg to about 260 mg, about 40 mg to about 240 mg, about 40 mg to about 220 mg, about 40 mg to about 200 mg, about 40 mg to about 180 mg, about 40 mg to about 160 mg, about 40 mg to about 140 mg, about 40 mg to about 120 mg, about 40 mg to about 100 mg, about 40 mg to about 80 mg, about 40 mg to about 60 mg, about 60 mg to about 400 mg, about 60 mg to about 380 mg, about 60 mg to about 360 mg, about 60 mg to about 340 mg, about 60 mg to about 320 mg, about 60 mg to about 300 mg, about 60 mg to about 280 mg, about 60 mg to about 260 mg, about 60 mg to about 240 mg, about 60 mg to about 220 mg, about 60 mg to about 200 mg, about 60 mg to about 180 mg, about 60 mg to about 160 mg, about 60 mg to about 140 mg, about 60 mg to about 120 mg, about 60 mg to about 100 mg, about 60 mg to about 80 mg, about 80 mg to about 400 mg, about 80 mg to about 380 mg, about 80 mg to about 360 mg, about 80 mg to about 340 mg, about 80 mg to about 320 mg, about 80 mg to about 300 mg, about 80 mg to about 280 mg, about 80 mg to about 260 mg, about 80 mg to about 240 mg, about 80 mg to about 220 mg, about 80 mg to about 200 mg, about 80 mg to about 180 mg, about 80 mg to about 160 mg, about 80 mg to about 140 mg, about 80 mg to about 120 mg, about 80 mg to about 100 mg, about 100 mg to about 400 mg, about 100 mg to about 380 mg, about 100 mg to about 360 mg, about 100 mg to about 340 mg, about 100 mg to about 320 mg, about 100 mg to about 300 mg, about 100 mg to about 280 mg, about 100 mg to about 260 mg, about 100 mg to about 240 mg, about 100 mg to about 220 mg, about 100 mg to about 200 mg, about 100 mg to about 180 mg, about 100 mg to about 160 mg, about 100 mg to about 140 mg, about 100 mg to about 120 mg, about 120 mg to about 400 mg, about 120 mg to about 380 mg, about 120 mg to about 360 mg, about 120 mg to about 340 mg, about 120 mg to about 320 mg, about 120 mg to about 300 mg, about 120 mg to about 280 mg, about 120 mg to about 260 mg, about 120 mg to about 240 mg, about 120 mg to about 220 mg, about 120 mg to about 200 mg, about 120 mg to about 180 mg, about 120 mg to about 160 mg, about 120 mg to about 140 mg, about 140 mg to about 400 mg, about 140 mg to about 380 mg, about 140 mg to about 360 mg, about 140 mg to about 340 mg, about 140 mg to about 320 mg, about 140 mg to about 300 mg, about 140 mg to about 280 mg, about 140 mg to about 260 mg, about 140 mg to about 240 mg, about 140 mg to about 220 mg, about 140 mg to about 200 mg, about 140 mg to about 180 mg, about 140 mg to about 160 mg, about 160 mg to about 400 mg, about 160 mg to about 380 mg, about 160 mg to about 360 mg, about 160 mg to about 360 mg, about 160 mg to about 340 mg, about 160 mg to about 320 mg, about 160 mg to about 300 mg, about 160 mg to about 280 mg, about 160 mg to about 260 mg, about 160 mg to about 240 mg, about 160 mg to about 220 mg, about 160 mg to about 200 mg, about 160 mg to about 180 mg, about 180 mg to about 400 mg, about 180 mg to about 380 mg, about 180 mg to about 360 mg, about 180 mg to about 340 mg, about 180 mg to about 320 mg, about 180 mg to about 300 mg, about 180 mg to about 280 mg, about 180 mg to about 260 mg, about 180 mg to about 240 mg, about 180 mg to about 220 mg, about 180 mg to about 200 mg, about 200 mg to about 400 mg, about 200 mg to about 380 mg, about 200 mg to about 360 mg, about 200 mg to about 340 mg, about 200 mg to about 320 mg, about 200 mg to about 300 mg, about 200 mg to about 280 mg, about 200 mg to about 260 mg, about 200 mg to about 240 mg, about 200 mg to about 220 mg, about 220 mg to about 400 mg, about 220 mg to about 380 mg, about 220 mg to about 360 mg, about 220 mg to about 340 mg, about 220 mg to about 320 mg, about 220 mg to about 300 mg, about 220 mg to about 280 mg, about 220 mg to about 260 mg, about 220 mg to about 240 mg, about 240 mg to about 400 mg, about 240 mg to about 380 mg, about 240 mg to about 360 mg, about 240 mg to about 340 mg, about 240 mg to about 320 mg, about 240 mg to about 300 mg, about 240 mg to about 280 mg, about 240 mg to about 260 mg, about 260 mg to about 400 mg, about 260 mg to about 380 mg, about 260 mg to about 360 mg, about 260 mg to about 340 mg, about 260 mg to about 320 mg, about 260 mg to about 300 mg, about 260 mg to about 280 mg, about 280 mg to about 400 mg, about 280 mg to about 380 mg, about 280 mg to about 360 mg, about 280 mg to about 340 mg, about 280 mg to about 320 mg, about 280 mg to about 300 mg, about 300 mg to about 400 mg, about 300 mg to about 380 mg, about 300 mg to about 360 mg, about 300 mg to about 340 mg, about 300 mg to about 320 mg, about 320 mg to about 400 mg, about 320 mg to about 380 mg, about 320 mg to about 360 mg, about 340 mg to about 360 mg, about 340 mg to about 400 mg, about 340 mg to about 380 mg, about 340 mg to about 360 mg, about 360 mg to about 400 mg, about 360 mg to about 380 mg, about 380 mg to about 400 mg, about 100 mg, about 200 mg, about 300 mg, or about 400 mg), and oral administration of a PARP inhibitor which is administered, for example once a day on a daily basis (during a period of time). In one embodiment, the KRAS inhibitor is orally administered once daily. In one embodiment, the KRAS inhibitor is orally administered twice daily.

One skilled in the art will recognize that, both in vivo and in vitro trials using suitable, known and generally accepted cell and/or animal models are predictive of the ability of a test compound of the combination or the combination to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### Synergy

In one embodiment, the addition of a PARP inhibitor, or a pharmaceutically acceptable salt or a pharmaceutical composition thereof, synergistically increases the activity of KRas G12C inhibitor compound of Formula (I), Formula I-A or Formula I-B against cancer or cancer cell lines expressing KRas G12C. Any method for determining whether two compounds exhibit synergy may be used for determining the synergistic effect of the combination.

Several mathematical models have been developed to determine whether two compounds act synergistically, i.e., beyond a mere additive effect. For instance, Loewe Additivity (Loewe (1928) Physiol. 27:47-187), Bliss Independence (Bliss (1939) Ann. Appl. Biol. 26:585-615), Highest Single Agent, ZIP (Yadav et al (2015) Comput Struct Biotech J 13:504-513) and other models (Chou & Talalay (1984) Adv Enzyme Regul 22:27-55. #6382953; and Greco et al. (1995) Pharmacol Rev 47 (2): 331-85. #7568331) are well known models in the pharmaceutical industry and may be used to calculate a “synergy score” that indicates whether synergy was detected and the magnitude of such synergy. Combining these synergy scores produces a composite synergy score which may be used to evaluate and characterize the KRas G12C inhibitor compounds of Formula (I), Formula I-A or Formula I-B in combination with a PARP inhibitor.

In general, the mathematical models use data obtained from single agent values to determine the predicted additive effect of the combination which is compared to the observed effect for the combination. If the observed effect is greater than the predicted effect, the combination is deemed to be synergistic. For example, the Bliss independence model compares the observed combination response (Yo) with the predicted combination response (YP), which was obtained based on the assumption that there is no effect from drug-drug interactions. Typically, the combination effect is declared synergistic if Yo is greater than YP.

In some embodiments, “synergistic effect” as used herein refers to combination of a KRAS inhibitor or a pharmaceutically acceptable salt thereof, and a PARP inhibitor or a pharmaceutically acceptable salt thereof producing an effect, for example, any of the beneficial or desired results including clinical results or endpoints as described herein, which is greater than the sum of the effect observed when a compound of Formula I or a pharmaceutically acceptable salt thereof (e.g., a compound selected from compound Nos. 1-678 (as numbered in WO2019099524), e.g., compound No. 234, 359, 478 or 507, or pharmaceutically acceptable salts thereof) and a PARP inhibitor or a pharmaceutically acceptable salt thereof are administered alone.

In some embodiments, the methods provided herein can result in a 1% to 99% (e.g., 1% to 98%, 1% to 95%, 1% to 90%, 1 to 85%, 1 to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 2% to 99%, 2% to 90%, 2% to 85%, 2% to 80%, 2% to 75%, 2% to 70%, 2% to 65%, 2% to 60%, 2% to 55%, 2% to 50%, 2% to 45%, 2% to 40%, 2% to 35%, 2% to 30%, 2% to 25%, 2% to 20%, 2% to 15%, 2% to 10%, 2% to 5%, 4% to 99%, 4% to 95%, 4% to 90%, 4% to 85%, 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10%, 6% to 99%, 6% to 95%, 6% to 90%, 6% to 85%, 6% to 80%, 6% to 75%, 6% to 70%, 6% to 65%, 6% to 60%, 6% to 55%, 6% to 50%, 6% to 45%, 6% to 40%, 6% to 35%, 6% to 30%, 6% to 25%, 6% to 20%, 6% to 15%, 6% to 10%, 8% to 99%, 8% to 95%, 8% to 90%, 8% to 85%, 8% to 80%, 8% to 75%, 8% to 70%, 8% to 65%, 8% to 60%, 8% to 55%, 8% to 50%, 8% to 45%, 8% to 40%, 8% to 35%, 8% to 30%, 8% to 25%, 8% to 20%, 8% to 15%, 10% to 99%, 10% to 95%, 10% to 90%, 10% to 85%, 10% to 80%, 10% to 75%, 10% to 70%, 10% to 65%, 10% to 60%, 10% to 55%, 10% to 50%, 10% to 45%, 10% to 40%, 10% to 35%, 10% to 30%, 10% to 25%, 10% to 20%, 10% to 15%, 15% to 99%, 15% to 95%, 15% to 90%, 15% to 85%, 15% to 80%, 15% to 75%, 15% to 70%, 15% to 65%, 15% to 60%, 15% to 55%, 15% to 50%, 15% to 55%, 15% to 50%, 15% to 45%, 15% to 40%, 15% to 35%, 15% to 30%, 15% to 25%, 15% to 20%, 20% to 99%, 20% to 95%, 20% to 90%, 20% to 85%, 20% to 80%, 20% to 75%, 20% to 70%, 20% to 65%, 20% to 60%, 20% to 55%, 20% to 50%, 20% to 45%, 20% to 40%, 20% to 35%, 20% to 30%, 20% to 25%, 25% to 99%, 25% to 95%, 25% to 90%, 25% to 85%, 25% to 80%, 25% to 75%, 25% to 70%, 25% to 65%, 25% to 60%, 25% to 55%, 25% to 50%, 25% to 45%, 25% to 40%, 25% to 35%, 25% to 30%, 30% to 99%, 30% to 95%, 30% to 90%, 30% to 85%, 30% to 80%, 30% to 75%, 30% to 70%, 30% to 65%, 30% to 60%, 30% to 55%, 30% to 50%, 30% to 45%, 30% to 40%, 30% to 35%, 35% to 99%, 35% to 95%, 35% to 90%, 35% to 85%, 35% to 80%, 35% to 75%, 35% to 70%, 35% to 65%, 35% to 60%, 35% to 55%, 35% to 50%, 35% to 45%, 35% to 40%, 40% to 99%, 40% to 95%, 40% to 90%, 40% to 85%, 40% to 80%, 40% to 75%, 40% to 70%, 40% to 65%, 40% to 60%, 40% to 55%, 40% to 60%, 40% to 55%, 40% to 50%, 40% to 45%, 45% to 99%, 45% to 95%, 45% to 95%, 45% to 90%, 45% to 85%, 45% to 80%, 45% to 75%, 45% to 70%, 45% to 65%, 45% to 60%, 45% to 55%, 45% to 50%, 50% to 99%, 50% to 95%, 50% to 90%, 50% to 85%, 50% to 80%, 50% to 75%, 50% to 70%, 50% to 65%, 50% to 60%, 50% to 55%, 55% to 99%, 55% to 95%, 55% to 90%, 55% to 85%, 55% to 80%, 55% to 75%, 55% to 70%, 55% to 65%, 55% to 60%, 60% to 99%, 60% to 95%, 60% to 90%, 60% to 85%, 60% to 80%, 60% to 75%, 60% to 70%, 60% to 65%, 65% to 99%, 60% to 95%, 60% to 90%, 60% to 85%, 60% to 80%, 60% to 75%, 60% to 70%, 60% to 65%, 70% to 99%, 70% to 95%, 70% to 90%, 70% to 85%, 70% to 80%, 70% to 75%, 75% to 99%, 75% to 95%, 75% to 90%, 75% to 85%, 75% to 80%, 80% to 99%, 80% to 95%, 80% to 90%, 80% to 85%, 85% to 99%, 85% to 95%, 85% to 90%, 90% to 99%, 90% to 95%, or 95% to 100%) reduction in the volume of one or more solid tumors in a patient following treatment with the combination therapy for a period of time between 1 day and 2 years (e.g., between 1 day and 22 months, between 1 day and 20 months, between 1 day and 18 months, between 1 day and 16 months, between 1 day and 14 months, between 1 day and 12 months, between 1 day and 10 months, between 1 day and 9 months, between 1 day and 8 months, between 1 day and 7 months, between 1 day and 6 months, between 1 day and 5 months, between 1 day and 4 months, between 1 day and 3 months, between 1 day and 2 months, between 1 day and 1 month, between one week and 2 years, between 1 week and 22 months, between 1 week and 20 months, between 1 week and 18 months, between 1 week and 16 months, between 1 week and 14 months, between 1 week and 12 months, between 1 week and 10 months, between 1 week and 9 months, between 1 week and 8 months, between 1 week and 7 months, between 1 week and 6 months, between 1 week and 5 months, between 1 week and 4 months, between 1 week and 3 months, between 1 week and 2 months, between 1 week and 1 month, between 2 weeks and 2 years, between 2 weeks and 22 months, between 2 weeks and 20 months, between 2 weeks and 18 months, between 2 weeks and 16 months, between 2 weeks and 14 months, between 2 weeks and 12 months, between 2 weeks and 10 months, between 2 weeks and 9 months, between 2 weeks and 8 months, between 2 weeks and 7 months, between 2 weeks and 6 months, between 2 weeks and 5 months, between 2 weeks and 4 months, between 2 weeks and 3 months, between 2 weeks and 2 months, between 2 weeks and 1 month, between 1 month and 2 years, between 1 month and 22 months, between 1 month and 20 months, between 1 month and 18 months, between 1 month and 16 months, between 1 month and 14 months, between 1 month and 12 months, between 1 month and 10 months, between 1 month and 9 months, between 1 month and 8 months, between 1 month and 7 months, between 1 month and 6 months, between 1 month and 6 months, between 1 month and 5 months, between 1 month and 4 months, between 1 month and 3 months, between 1 month and 2 months, between 2 months and 2 years, between 2 months and 22 months, between 2 months and 20 months, between 2 months and 18 months, between 2 months and 16 months, between 2 months and 14 months, between 2 months and 12 months, between 2 months and 10 months, between 2 months and 9 months, between 2 months and 8 months, between 2 months and 7 months, between 2 months and 6 months, or between 2 months and 5 months, between 2 months and 4 months, between 3 months and 2 years, between 3 months and 22 months, between 3 months and 20 months, between 3 months and 18 months, between 3 months and 16 months, between 3 months and 14 months, between 3 months and 12 months, between 3 months and 10 months, between 3 months and 8 months, between 3 months and 6 months, between 4 months and 2 years, between 4 months and 22 months, between 4 months and 20 months, between 4 months and 18 months, between 4 months and 16 months, between 4 months and 14 months, between 4 months and 12 months, between 4 months and 10 months, between 4 months and 8 months, between 4 months and 6 months, between 6 months and 2 years, between 6 months and 22 months, between 6 months and 20 months, between 6 months and 18 months, between 6 months and 16 months, between 6 months and 14 months, between 6 months and 12 months, between 6 months and 10 months, or between 6 months and 8 months) (e.g., as compared to the size of the one or more solid tumors in the patient prior to treatment).

The phrase “time of survival” means the length of time between the identification or diagnosis of cancer (e.g., any of the cancers described herein) in a mammal by a medical professional and the time of death of the mammal (caused by the cancer). Methods of increasing the time of survival in a mammal having a cancer are described herein.

In some embodiments, any of the methods described herein can result in an increase (e.g., a 1% to 400%, 1% to 380%, 1% to 360%, 1% to 340%, 1% to 320%, 1% to 300%, 1% to 280%, 1% to 260%, 1% to 240%, 1% to 220%, 1% to 200%, 1% to 180%, 1% to 160%, 1% to 140%, 1% to 120%, 1% to 100%, 1% to 95%, 1% to 90%, 1% to 85%, 1% to 80%, 1% to 75%, 1% to 70%, 1% to 65%, 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 5% to 400%, 5% to 380%, 5% to 360%, 5% to 340%, 5% to 320%, 5% to 300%, 5% to 280%, 5% to 260%, 5% to 240%, 5% to 220%, 5% to 200%, 5% to 180%, 5% to 160%, 5% to 140%, 5% to 120%, 5% to 100%, 5% to 90%, 5% to 80%, 5% to 70%, 5% to 60%, 5% to 50%, 5% to 40%, 5% to 30%, 5% to 20%, 5% to 10%, 10% to 400%, 10% to 380%, 10% to 360%, 10% to 340%, 10% to 320%, 10% to 300%, 10% to 280%, 10% to 260%, 10% to 240%, 10% to 220%, 10% to 200%, 10% to 180%, 10% to 160%, 10% to 140%, 10% to 120%, 10% to 100%, 10% to 90%, 10% to 80%, 10% to 70%, 10% to 60%, 10% to 50%, 10% to 40%, 10% to 30%, 10% to 20%, 20% to 400%, 20% to 380%, 20% to 360%, 20% to 340%, 20% to 320%, 20% to 300%, 20% to 280%, 20% to 260%, 20% to 240%, 20% to 220%, 20% to 200%, 20% to 180%, 20% to 160%, 20% to 140%, 20% to 120%, 20% to 100%, 20% to 90%, 20% to 80%, 20% to 70%, 20% to 60%, 20% to 50%, 20% to 40%, 20% to 30%, 30% to 400%, 30% to 380%, 30% to 360%, 30% to 340%, 30% to 320%, 30% to 300%, 30% to 280%, 30% to 260%, 30% to 240%, 30% to 220%, 30% to 200%, 30% to 180%, 30% to 160%, 30% to 140%, 30% to 120%, 30% to 100%, 30% to 90%, 30% to 80%, 30% to 70%, 30% to 60%, 30% to 50%, 30% to 40%, 40% to 400%, 40% to 380%, 40% to 360%, 40% to 340%, 40% to 320%, 40% to 300%, 40% to 280%, 40% to 260%, 40% to 240%, 40% to 220%, 40% to 200%, 40% to 180%, 40% to 160%, 40% to 140%, 40% to 120%, 40% to 100%, 40% to 90%, 40% to 80%, 40% to 70%, 40% to 60%, 40% to 50%, 50% to 400%, 50% to 380%, 50% to 360%, 50% to 340%, 50% to 320%, 50% to 300%, 50% to 280%, 50% to 260%, 50% to 240%, 50% to 220%, 50% to 200%, 50% to 180%, 50% to 160%, 50% to 140%, 50% to 140%, 50% to 120%, 50% to 100%, 50% to 90%, 50% to 80%, 50% to 70%, 50% to 60%, 60% to 400%, 60% to 380%, 60% to 360%, 60% to 340%, 60% to 320%, 60% to 300%, 60% to 280%, 60% to 260%, 60% to 240%, 60% to 220%, 60% to 200%, 60% to 180%, 60% to 160%, 60% to 140%, 60% to 120%, 60% to 100%, 60% to 90%, 60% to 80%, 60% to 70%, 70% to 400%, 70% to 380%, 70% to 360%, 70% to 340%, 70% to 320%, 70% to 300%, 70% to 280%, 70% to 260%, 70% to 240%, 70% to 220%, 70% to 200%, 70% to 180%, 70% to 160%, 70% to 140%, 70% to 120%, to 100%, 70% to 90%, 70% to 80%, 80% to 400%, 80% to 380%, 80% to 360%, 80% to 340%, 80% to 320%, 80% to 300%, 80% to 280%, 80% to 260%, 80% to 240%, 80% to 220%, 80% to 200%, 80% to 180%, 80% to 160%, 80% to 140%, 80% to 120%, 80% to 100%, 80% to 90%, 90% to 400%, 90% to 380%, 90% to 360%, 90% to 340%, 90% to 320%, 90% to 300%, 90% to 280%, 90% to 260%, 90% to 240%, 90% to 220%, 90% to 200%, 90% to 180%, 90% to 160%, 90% to 140%, 90% to 120%, 90% to 100%, 100% to 400%, 100% to 380%, 100% to 360%, 100% to 340%, 100% to 320%, 100% to 300%, 100% to 280%, 100% to 260%, 100% to 240%, 100% to 220%, 100% to 200%, 100% to 180%, 100% to 160%, 100% to 140%, 100% to 120%, 120% to 400%, 120% to 380%, 120% to 360%, 120% to 340%, 120% to 320%, 120% to 300%, 120% to 280%, 120% to 260%, 120% to 240%, 120% to 220%, 120% to 200%, 120% to 180%, 120% to 160%, 120% to 140%, 140% to 400%, 140% to 380%, 140% to 360%, 140% to 340%, 140% to 320%, 140% to 300%, 140% to 280%, 140% to 260%, 140% to 240%, 140% to 220%, 140% to 200%, 140% to 180%, 140% to 160%, 160% to 400%, 160% to 380%, 160% to 360%, 160% to 340%, 160% to 320%, 160% to 300%, 160% to 280%, 160% to 260%, 160% to 240%, 160% to 220%, 160% to 200%, 160% to 180%, 180% to 400%, 180% to 380%, 180% to 360%, 180% to 340%, 180% to 320%, 180% to 300%, 180% to 280%, 180% to 260%, 180% to 240%, 180% to 220%, 180% to 200%, 200% to 400%, 200% to 380%, 200% to 360%, 200% to 340%, 200% to 320%, 200% to 300%, 200% to 280%, 200% to 260%, 200% to 240%, 200% to 220%, 220% to 400%, 220% to 380%, 220% to 360%, 220% to 340%, 220% to 320%, 220% to 300%, 220% to 280%, 220% to 260%, 220% to 240%, 240% to 400%, 240% to 380%, 240% to 360%, 240% to 340%, 240% to 320%, 240% to 300%, 240% to 280%, 240% to 260%, 260% to 400%, 260% to 380%, 260% to 360%, 260% to 340%, 260% to 320%, 260% to 300%, 260% to 280%, 280% to 400%, 280% to 380%, 280% to 360%, 280% to 340%, 280% to 320%, 280% to 300%, 300% to 400%, 300% to 380%, 300% to 360%, 300% to 340%, or 300% to 320%) in the time of survival of the patient (e.g., as compared to a patient having a similar cancer and administered a different treatment or not receiving a treatment).

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

### Kits

The present invention also relates to a kit comprising a PARP inhibitor, or a pharmaceutically acceptable salt or a pharmaceutical composition thereof, and a KRas G12C inhibitor compound of Formula (I), Formula I-A or Formula I-B, or a pharmaceutically acceptable salt or a pharmaceutical composition thereof. Also provided is a kit comprising a PARP inhibitor, or a pharmaceutically acceptable salt or a pharmaceutical composition thereof, and a KRas G12C inhibitor compound of Formula (I), Formula I-A or Formula I-B, or a pharmaceutically acceptable salt or a pharmaceutical composition thereof, for use in treating a hematological cancer.

In a related aspect, the invention provides a kit containing a dose of a PARP inhibitor, or a pharmaceutically acceptable salt or a pharmaceutical composition thereof, and dose of a KRas G12C inhibitor compound of Formula (I), Formula I-A or Formula I-B, or a pharmaceutically acceptable salt or a pharmaceutical composition thereof, in an amount effective to inhibit proliferation of cancer cells, particularly KRas G12C-expressing cancer cells, in a subject. The kit in some cases includes an insert with instructions for administration of the a PARP inhibitor, or a pharmaceutically acceptable salt or a pharmaceutical composition thereof, and a KRas G12C inhibitor compound of Formula (I), Formula I-A or Formula I-B, or a pharmaceutically acceptable salt or a pharmaceutical composition thereof. The insert may provide a user with one set of instructions for using the a PARP inhibitor, or a pharmaceutically acceptable salt or a pharmaceutical composition thereof, in combination with a KRas G12C inhibitor compound of Formula (I), Formula I-A or Formula I-B, or a pharmaceutically acceptable salt or a pharmaceutical composition thereof.

### Example A

### PARP Inhibitors Increase the Activity of KRas G12C Inhibitors Against Cell Lines Expressing KRas G12C

This Example illustrates that the combination of a a PARP inhibitor and exemplary KRas G12C inhibitor compounds of Formula I, Formula I-A and Formula 1-B synergistically inhibits the growth of tumor cell lines that express KRas G12C.

Two lung cancer cell lines harboring KRas G12C mutations was assembled to determine whether combining PARP inhibitors with exemplary KRas G12C inhibitors disclosed herein results in synergistic activity. The collection included NCI-H2030 (ATCC CRL-5985) and NCI-H2122 (ATCC CRL-5985).

Assays for determining the synergy score for the pairwise combinations for each cell line were performed in triplicate. Three 96-well plates plus an additional 4 wells of a separate 96-well control plate for determining baseline luminescence were seeded with 5000 cells/well of a particular cell line in a total volume of 90 μl of a suitable growth medium for that cell line, e.g., RPMI 1640 medium supplemented with 10% FBS and any cell line specific reagents need for growth. The plates were incubated overnight at 37° C. in a 5% CO₂ atmosphere.

To each of the designated baseline wells, 30 μl of Cell-Titer Glo reagent (CTG; Promega Corporation) was added to each well and the plates were incubated for 20 min with shaking at room temperature. Baseline luminescence was quantitated using a BMG ClarioStar multimode plate reader according to the manufacturer's instructions.

A series of working stock 1000× drug dilutions in 100% DMSO was prepared that includes a 9 point single agent dilution of the exemplary KRas G12C inhibitor of Formula (I), Formula I-A and Formula I-B and a 5-point single agent dilution of the PARP inhibitor. The dilutions used for the KRas G12C inhibitor and the PARP inhibitor varied for each individual compound but were in the range of 3- to 6-fold/serial dilution.

Exemplary KRas G12C inhibitors tested in this Example included:

| Example | |
|---|---|
| No.* | Structure |
| 234 | 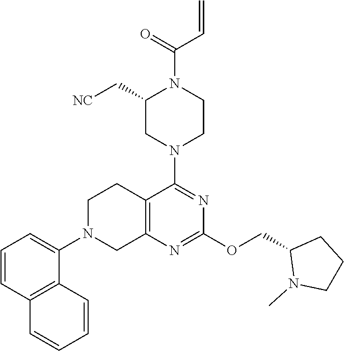 [SMILES: C=CC(=O)N1CCN(c2nc(OC[C@@H]3CCCN3C)nc3c2CCN(c2cccc4ccccc24)C3)C[C@@H]1CC#N] |
| 359 | 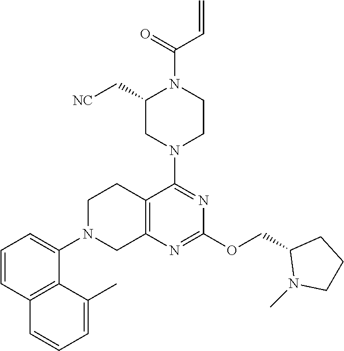 [SMILES: C=CC(=O)N1CCN(c2nc(OC[C@@H]3CCCN3C)nc3c2CCN(c2cccc4cccc(C)c24)C3)C[C@@H]1CC#N] |
| 478 | 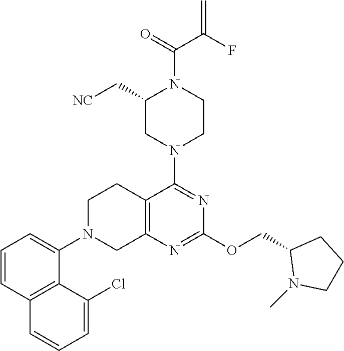 [SMILES: C=C(F)C(=O)N1CCN(c2nc(OC[C@@H]3CCCN3C)nc3c2CCN(c2cccc4cccc(Cl)c24)C3)C[C@@H]1CC#N] |
| 507 | 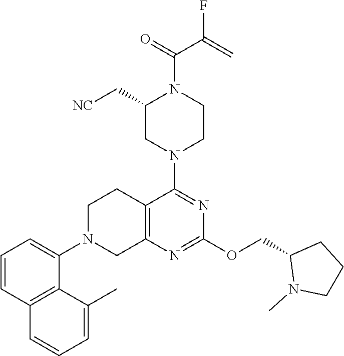 [SMILES: C=C(F)C(=O)N1CCN(c2nc(OC[C@@H]3CCCN3C)nc3c2CCN(c2cccc4cccc(C)c24)C3)C[C@@H]1CC#N] |

*Example Number refers to the example number for each compound as
disclosed in published international PCT application publication number
WO2019099524.

A 10× intermediate dosing plate was prepared in serum free RPMI medium that contains arrayed single agent dilutions of exemplary KRas G12C inhibitor of Formula (I) or the PARP inhibitor. In addition, a matrix of 40 dilution combinations of exemplary KRas G12C inhibitor of Formula (I), Formula I-A or Formula I-B and the PARP inhibitor was prepared as test samples.

To each corresponding well of the three 96-well plates seeded with the appropriate cell line above, 10 μl of each 10× single agent and the 40 combinations of the dose matrix was added and the plates were incubated for 72 hours at 37C in 5% CO₂ atmosphere. A 30 μl aliquot of Cell-Titer Glo reagent (CTG) was added to each test well, the plates were incubated for 20 min with shaking at room temperature, and luminescence was quantitated using a BMG ClarioStar multimode plate reader according to the manufacturer's instructions.

The raw data and metadata files were used as input files to calculate percent effect for each treatment condition and analyzed using four independent mathematical reference models designed to determine whether the two test compounds demonstrate synergy: Loewe additivity, Bliss independence, Highest Single Agent and ZIP.

The output of the data from each mathematical model is the assignment of a relative synergy score. The data reported in Table 1A and Table 1B are the aggregate sum of the Loewe additivity, Bliss independence, Highest Single Agent and ZIP scores (“Composite Synergy Score”). A positive score indicates synergy between two or more compounds, whereas a negative score indicates a lack of synergy. Greater positive values indicate higher synergy between two or more compounds.

TABLE 1A
Composite Synergy Scores for an Exemplary PARP
Inhibitors Combined with Exemplary KRas G12C
Inhibitor MRTX849Against KRas G12C Cell Lines

| | PARP Inhibitor | | | |
|---|---|---|---|---|
| | olaparib | talazoparib | veliparib | rucaparib |
| | KRas G12C (Example #) | | | |
| | MRTX849 | MRTX849 | MRTX849 | MRTX849 |
| Cell Line | (478) | (478) | (478) | (478) |
| H2030 | 5 | 21 | −13 | −5 |
| H2122 | 11 | 27 | 23 | 23 |

TABLE 1B
Composite Synergy Scores for Exemplary PARP Inhibitors
Combined with Exemplary KRas G12C Inhibitors of Formula
(I), Formula I-A and Formula I-B Against KRas G12C
Cell Lines Using Hypoxic or Normal Conditions

PARP Inhibitor

| | |
|---|---|
| olaparib | talazoparib |

KRas G12C (Example #)

| | | MRTX849 | MRTX849 |
|---|---|---|---|
| Cell Line | Condition | (478) | (478) |
| H1373 | Hypoxia | 10 | 6 |
| | Normoxia | 7 | 9 |
| LU99 | Hypoxia | 19 | −1 |
| | Normoxia | 14 | 14 |

These results demonstrate that the panel of KRas G12C cell lines exhibit a modest synergistic effect for the combination of a PARP inhibitor with exemplary KRas G12C inhibitor MRTX849 (example 478 of WO2019/099524) in certain cell lines warranting further interrogation of the combine efficacy studies in in vivo models.

### Example B

### In Vivo Models for Examining KRas G12C Inhibitor Plus PARP Inhibitor Combinations

Immunocompromised nude/nude mice were inoculated in the right hind flank with cells or patient derived tumor samples harboring a KRas G12C mutation. When tumor volumes reach ˜300 mm³ in size, the mice were divided into four groups of 5 mice each. The first group was administered vehicle only. The second group was orally administered a single agent dose of the KRas G12C inhibitor daily at a concentration that yields a maximal biological effect that does not result in complete tumor regression. The third group was orally administered a single agent dose of the PARP inhibitor daily at a concentration that yields a maximal biological effect that also does not result in complete tumor regression. The fourth group was orally administered the single agent dose of the KRas G12C inhibitor in combination with the single agent dose of the PARP inhibitor at the matched schedule. The treatment period was 17 days. Tumor volumes were measured using a caliper every two-three days and tumor volumes are calculated by the formula: 0.5×(Length×Width)². A greater degree of tumor growth inhibition for the combination in this model demonstrated that the combination therapy is likely to have a clinically meaningful benefit to treated subjects relative to treatment with only a KRas G12C inhibitor.

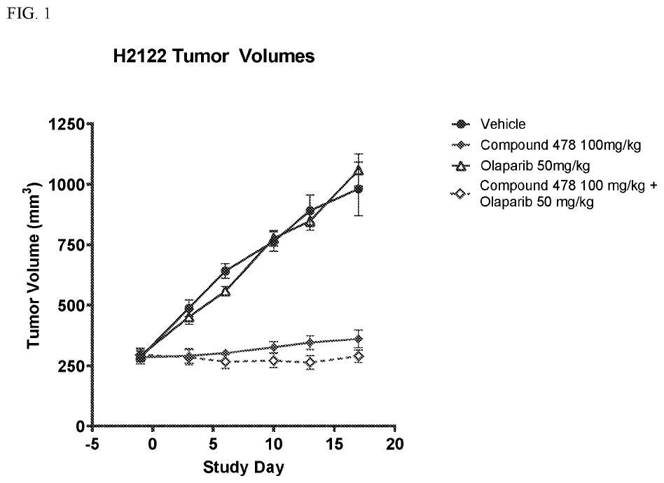

For example, twenty nude/nude mice were inoculated in the right hind limb with 5×10⁶ H2122 cells. When tumor volume reached ˜300 mm³ (Study Day 1), 5 mice in each of the five groups were administered p.o. daily for 32 days: vehicle only (10% Captisol in 50 mM citrate buffer pH5.0), 100 mg/kg of KRas G12C inhibitor Compound 478 (10% Captisol in 50 mM citrate buffer, pH 5.0), 50 mg/kg of the PARP inhibitor olaparib (10% Captisol in 50 mM citrate buffer, pH 5.0), or 100 mg/kg of KRas G12C inhibitor Compound 478 and 50 mg/kg of olaparib. Dosing stopped on day 17 for all groups due to large tumor growth in the vehicle and Olaparib only groups. Tumor volumes, measured at pre-specified days, for the five mice per group were averaged and are reported in FIG. 1 and Table 2.

TABLE 2
Average Tumor Volumes (mm³) of H2122 Tumor Bearing
Mice Treated with Single Agents and in Combination

| | | | | Compound 478 + |
|---|---|---|---|---|
| Study | | Compound | | Olaparib |
| Day | Vehicle | 478 | Olaparib | Combination |
| −1 | 284.728 | 284.794 | 292.194 | 294.492 |
| 3 | 487.382 | 290.578 | 451.342 | 284.33 |
| 6 | 641.168 | 301.744 | 558.15 | 266.928 |
| 10 | 763.78 | 326.222 | 776.514 | 271.178 |
| 13 | 890.67 | 345.38 | 848.664 | 263.13 |
| 17 | 980.99 | 360.308 | 1058.96 | 289.524 |

As shown in FIG. 1 and Table 2, the administration of olaparib as a single agent exhibited 0% tumor growth inhibition on Day 17 while the administration of KRas G12C inhibitor Compound 478 as a single agent exhibited 89% tumor growth inhibition. The combination of the PARP inhibitor olaparib and Compound 478, however, resulted in 2% tumor regression at Day 17.

These results demonstrate that the combination therapy resulted in greater amount of tumor growth regression compared to either single agent alone in the H2122 cell-line derived xenograft model and with a PARP inhibitor, demonstrating enhanced in vivo anti-tumor efficacy of the combination against KRas G12C expressing cancer.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1-58. (canceled)

59. A method of treating cancer in a subject in need thereof, comprising administering to the subject a therapeutically effective amount of a combination of PARP inhibitor and a KRAS G12C inhibitor of formula: 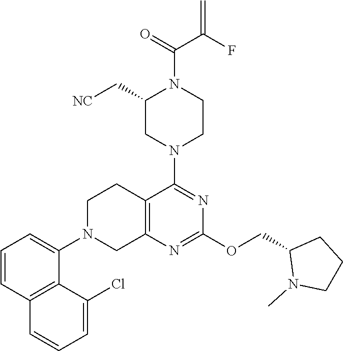 [SMILES: C=C(F)C(=O)N1CCN(c2nc(OC[C@@H]3CCCN3C)nc3c2CCN(c2cccc4cccc(Cl)c24)C3)C[C@@H]1CC#N]
or a pharmaceutically acceptable salt thereof.

60. The method according to claim 59, wherein the PARP inhibitor is selected from the group consisting of olaparib, rucaparib, niraparib, talazoparib, veliparib, RBN-2397, I-1, KMR-206, RP14042 and JAB-26766.

61. The method of claim 59, wherein the PARP inhibitor is olaparib.

62. The method of claim 59, wherein the PARP inhibitor is rucaparib.

63. The method of claim 59, wherein the PARP inhibitor is niraparib.

64. The method of claim 59, wherein the PARP inhibitor is talazoparib.

65. The method of claim 59, wherein the PARP inhibitor is veliparib.

66. The method according to claim 59, wherein the PARP inhibitor and the KRAS G12C inhibitor are administered on the same day.

67. The method according to claim 59, wherein the PARP inhibitor and the KRAS G12C inhibitor are administered on different days.

68. The methos according to claim 59, wherein the KRas G12C inhibitor is administered at a maximum tolerated dose.

69. The method according to claim 59, wherein the PARP inhibitor and the KRAS G12C inhibitor are each administered at a maximum tolerated dose.

70. The method according to claim 59, wherein the therapeutically effective amount of the combination of the PARP inhibitor and the KRAS G12C inhibitor results in an increased duration of overall survival, an increased duration of progression free survival, an increase in tumor growth regression, an increase in tumor growth inhibition or an increased duration of stable disease in the subjects relative to treatment with only the KRas G12C inhibitor.

71. A pharmaceutical composition, comprising a therapeutically effective amount of a combination of a PARP inhibitor and a KRas G12 inhibitor of formula: 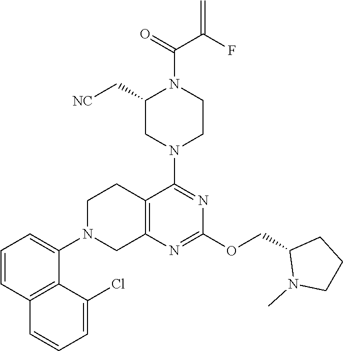 [SMILES: C=C(F)C(=O)N1CCN(c2nc(OC[C@@H]3CCCN3C)nc3c2CCN(c2cccc4cccc(Cl)c24)C3)C[C@@H]1CC#N]
or a pharmaceutically acceptable salt thereof, and a pharmaceutically acceptable excipient.

72. The pharmaceutical composition of claim 71, wherein the PARP inhibitor is selected from the group consisting of olaparib, rucaparib, niraparib, talazoparib, and veliparib.

73. A method for inhibiting KRas G12C activity in a cell, comprising contacting the cell in which inhibition of KRas G12C activity is desired with an effective amount of a PARP inhibitor, or a pharmaceutical composition or a pharmaceutically acceptable salt thereof, and a KRas G12C inhibitor of formula: 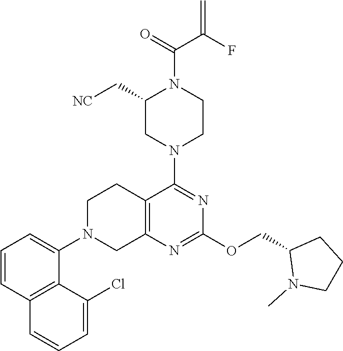 [SMILES: C=C(F)C(=O)N1CCN(c2nc(OC[C@@H]3CCCN3C)nc3c2CCN(c2cccc4cccc(Cl)c24)C3)C[C@@H]1CC#N]
and a pharmaceutically acceptable salt thereof, wherein the PARP inhibitor synergistically increases the sensitivity of the cancer cells to the KRas G12C inhibitor.

74. A method according to claim 73, wherein the PARP inhibitor synergistically increases the sensitivity of the cancer cells to the KRas G12C inhibitor.

75. The method according to claim 73, wherein the therapeutically effective amount of the KRas G12C inhibitor in the combination is between about 0.01 to 100 mg/kg per day.

76. The method of claim 75, wherein the therapeutically effective amount of the KRas G12C inhibitor in the combination is between about 0.1 to 50 mg/kg per day.

77. The method according to claim 75, wherein the therapeutically effective amount of the PARP inhibitor in the combination is between about 0.01 to 100 mg/kg per day.

78. The method of claim 77, wherein the therapeutically effective amount of the PARP inhibitor in the combination is between about 0.1 to 50 mg/kg per day.

79. The method of any of claim 59, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial ‘carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

80. The method of claim 79, wherein the cancer wherein the cancer is a KRas G12C-associated cancer.

81. The method of claim 79, wherein the cancer is non-small cell lung cancer.

82. The method of claim 79, wherein the cancer is bladder cancer.

83. The method of claim 79, wherein the cancer is cervical cancer.

84. The method of claim 79, wherein the cancer is colorectal cancer.

85. A kit comprising the pharmaceutical composition of claim 59, for treating KRas G12C cancer in a subject.

86. A kit comprising: a) a pharmaceutical composition comprising a PARP inhibitor and b) a pharmaceutical composition comprising a KRas G12C inhibitor of formula: 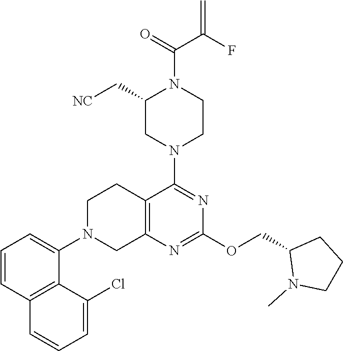 [SMILES: C=C(F)C(=O)N1CCN(c2nc(OC[C@@H]3CCCN3C)nc3c2CCN(c2cccc4cccc(Cl)c24)C3)C[C@@H]1CC#N]
and a pharmaceutically acceptable salt thereof.

87. The kit according to claim 86, wherein the PARP inhibitor is selected from the group consisting of olaparib, rucaparib, niraparib, talazoparib, and veliparib.

88. The kit according to claim 86, further comprising an insert with instructions for administration of one or both pharmaceutical compositions.
